SEQUENCE LISTING (54 sequences)

SEQ ID NO: 1-32 - skipped (32 entries)

SEQ ID NO: 33 - DNA, 12 bp, synthetic construct
  source 1..12: mol_type = other DNA
gacaaaaaaaaa

SEQ ID NO: 34 - DNA, 12 bp, synthetic construct
  source 1..12: mol_type = other DNA
tccaaaaaaaaa

SEQ ID NO: 35 - DNA, 12 bp, synthetic construct
  source 1..12: mol_type = other DNA
tggaaaaaaaaa

SEQ ID NO: 36 - DNA, 12 bp, synthetic construct
  source 1..12: mol_type = other DNA
aactaaaaaaaa

SEQ ID NO: 37 - DNA, 12 bp, synthetic construct
  source 1..12: mol_type = other DNA
agttaaaaaaaa

SEQ ID NO: 38 - DNA, 12 bp, synthetic construct
  source 1..12: mol_type = other DNA
ttcgaaaaaaaa

SEQ ID NO: 39 - DNA, 12 bp, synthetic construct
  source 1..12: mol_type = other DNA
cgaaaaaaaaaa

SEQ ID NO: 40 - DNA, 12 bp, synthetic construct
  source 1..12: mol_type = other DNA
aatgaaaaaaaa

SEQ ID NO: 41 - DNA, 12 bp, synthetic construct
  source 1..12: mol_type = other DNA
cattaaaaaaaa

SEQ ID NO: 42 - DNA, 12 bp, synthetic construct
  source 1..12: mol_type = other DNA
agacaaaaaaaa

SEQ ID NO: 43 - DNA, 12 bp, synthetic construct
  source 1..12: mol_type = other DNA
gtctaaaaaaaa

SEQ ID NO: 44 - DNA, 12 bp, synthetic construct
  source 1..12: mol_type = other DNA
agcgaaaaaaaa

SEQ ID NO: 45 - DNA, 12 bp, synthetic construct
  source 1..12: mol_type = other DNA
cgctaaaaaaaa

SEQ ID NO: 46 - DNA, 12 bp, synthetic construct
  source 1..12: mol_type = other DNA
gtataaaaaaaa

SEQ ID NO: 47 - DNA, 12 bp, synthetic construct
  source 1..12: mol_type = other DNA
atacaaaaaaaa

SEQ ID NO: 48 - DNA, 12 bp, synthetic construct
  source 1..12: mol_type = other DNA
tagcaaaaaaaa

SEQ ID NO: 49 - DNA, 20 bp, synthetic construct
  source 1..20: mol_type = other DNA
  variation 1..4: note = n is one of A, G, C, T or U
nnnnaaaaaaaaaaaaaaaa

SEQ ID NO: 50 - RNA, 20 nt, synthetic construct
  source 1..20: mol_type = other RNA
  variation 1..4: note = n is one of A, G, C, T or U
nnnnaaaaaaaaaaaaaaaa

SEQ ID NO: 51 - DNA, 16 bp, synthetic construct
  source 1..16: mol_type = other DNA
tgtctactcaacctgt

SEQ ID NO: 52-54 - skipped (3 entries)

(19) United States
(12) Patent Application Publication
(10) Pub. No.: US 2026/0209813 A1
(43) Pub. Date: Jul. 23, 2026

(54) LIGATION OF OLIGONUCLEOTIDES

(71) Applicant: Ribbon Bio GmbH, Vienna (AT)

(72) Inventors: Luca AGOZZINO, Vienna (AT); Marc Jochen BREHME, Vienna (AT)

(21) Appl. No.: 19/141,185

(22) PCT Filed: Dec. 20, 2022
(86) PCT No.: PCT/EP2022/086980

§ 371 Date: Jun. 19, 2025

(51) Int. Cl. C12P 19/34

(52) U.S. Cl. CPC C12P 19/34

(57) ABSTRACT

A method to produce a target double-stranded (ds) DNA polynucleotide by annealing single-stranded oligonucleotides (ss oligos) and ligating a ds assembly, comprising the following steps: a) annealing at least 2 ss oligos comprising matching sequences for hybridization to produce a starter ds oligo with two overhangs, and hybridizing one of said overhangs with a nucleotide (NA) building block, preferably any one of a ss oligo, ds oligo or ds polynucleotide, thereby obtaining a ds assembly with two overhangs of at least 2 nucleotides (nt), which ds assembly comprises at least one nick in the ds part of the ds assembly; and b) hybridizing to the ds assembly at least one extender ss oligo (ExtON), which comprises a sticky terminal sequence of at least 2 nt on one side, and an identifiable tail on the other side, which sticky sequence is matching for hybridization with an overhang of the ds assembly, thereby obtaining an extended ds assembly; and c) ligating the nucleotides within the extended ds assembly by a ligation reaction, thereby resolving said at least one nick; and d) removing said at least one ExtON from the extended ds assembly upon said ligation reaction, thereby producing the target ds polynucleotide.

### FIELD OF THE INVENTION

The invention relates to a method of synthesizing a double stranded target DNA polynucleotide by annealing and ligating single-stranded oligonucleotides.

### BACKGROUND OF THE INVENTION

A growing demand for DNA oligonucleotides (oligos) and polynucleotides in the biological, biotechnological, chemical or medical sector requires a high-throughput, cost-efficient and flexible synthesis of oligonucleotides and polynucleotides in high quality.

Artificial synthesis of polynucleotides can be achieved through biochemical “assembly methods”. Assembling is started by complex formation (annealing) and base-pairing via hydrogen bonds (hybridization) of complementary single stranded (ss) oligos to yield the respective double-stranded (ds) oligos. To join ds oligos, a ligase enzyme is commonly used. By a ligase reaction, a nick between the ds oligos to be joined can be closed, yielding a DNA ligation product. In particular, DNA ligases produce a covalent phosphodiester bond between two nucleic acids, one carrying a 5′ phosphate group and the other a 3′ hydroxyl group. Intermediate oligonucleotides and polynucleotides of different sizes can be further ligated with others, in order to obtain a larger molecule that has the desired target sequence.

There are indications that an efficient ligation by ligase depends on oligonucleotide length, as ligases make specific contacts over a length of DNA to stabilise the DNA-protein complex. T4 DNA ligase is particularly useful in DNA synthesis, since the enzyme catalyses the ligation of duplex DNA strands with both overhangs and blunt ends.

Horspool et al. (2010) used immobilized ds DNA on beads, wherein the free end is designed with a variable 5′ overhang, to determine the minimum template overlap and minimum oligo length necessary for efficient ligation of a complementary 5′-phosphorylated ss DNA fragment by T4 DNA ligase. Ligation by T4 DNA ligase was found to be dependent on the formation of a ds DNA duplex of at least 5 base pairs surrounding a nick. Supplementary ds oligos were used to extend the immobilized duplex for ligation.

WO2019073072A1 and WO2020208234A1 disclose a DNA assembly approach starting from ss oligos forming ds oligos with an overhang as an intermediate product to produce a longer ds polynucleotide.

Although the last few years have seen considerable progress in the techniques for synthesizing DNA, there are still restrictions in DNA assemblies in particular where short ss oligos are used.

### SUMMARY OF THE INVENTION

It is an objective of the present invention to provide a tool and a method for synthesizing double stranded (ds) DNA, aiming to ligate an assembly comprising short single stranded (ss) oligonucleotides (oligos).

The objective is solved by the subject of the present claims and as further described herein.

The invention provides for a method to produce a target double-stranded (ds) DNA polynucleotide by annealing single-stranded oligonucleotides (ss oligos) and ligating a ds assembly, comprising the following steps:
a) annealing at least 2 ss oligos comprising matching sequences for hybridization to produce a starter ds oligo with two overhangs, and hybridizing one of said overhangs with a nucleotide (NA) building block, preferably any one of a ss oligo, ds oligo or ds polynucleotide, thereby obtaining a ds assembly with two overhangs of at least 2 nucleotides (nt), which ds assembly comprises at least one nick in the ds part of the ds assembly; and
b) hybridizing to the ds assembly at least one extender ss oligo (ExtON), which comprises a sticky terminal sequence of at least 2 nt on one side, and an identifiable tail on the other side, which sticky sequence is matching for hybridization with an overhang of the ds assembly, thereby obtaining an extended ds assembly; and
c) ligating the nucleotides within the extended ds assembly, by a ligation reaction, thereby resolving said at least one nick; and
d) removing said at least one ExtON from the extended ds assembly upon said ligation reaction, thereby producing the target ds polynucleotide.

Specifically, in step a), the NA building block hybridized to the starter ds oligo is any one of a ss oligo, ds oligo or ds polynucleotide.

Where the NA building block added to the starter assembly is a ss oligo, at least three ss oligos can be annealed to obtain the starter ds oligo that can be used as a ds assembly in the method described herein. Because either of the ss oligos used for annealing in step a) of the method described herein, is understood to be a NA building block, the ds assembly can be produced by adding at least one further ss oligo as further NA building block(s) for annealing and/or hybridization.

According to a specific aspect, in step a):
i) at least four ss oligos are annealed and hybridized to produce a ds assembly comprising at least two nicks; or
ii) three ss oligos are annealed and hybridized to produce a ds assembly comprising one nick;
iii) two ss oligos are annealed and hybridized to one ds oligo or ds polynucleotide to produce a ds assembly comprising one or two nicks.

Specifically, in step a) of the method described herein, annealing a number of ss oligos (herein also referred to as source oligos or source ss oligos) produces a starter ds oligo (which is herein also referred to as a starter assembly) or a ds assembly.

Specifically, the number of source ss oligos is any one of 3, 4, 5, or 6 ss oligos.

Specifically, the length of ss oligos used as source ss oligos to produce a starter ds oligo or ds assembly as described herein, can be the same or different e.g., ranging between 4 and 12 nt e.g., 4, 5, 6, 7, 8, 9, 10, 11, or 12 nt, preferably 6 nt or 8 nt.

Specifically, one or more or each of the ss oligos comprises or consists of at least any one of 6, 7, 8, 9, 10, 11, or 12 nt.

According to a specific aspect, each of the ss oligos comprises or consists of at least 6 nt or at least 8 nt.

Specifically, one or more or each of the ss oligos comprises or consists of up to any one of 20, 19, 18, 17, 16, 15, 14, 13, 12, 11, 10, 9, 8, 7, or 6 nt, preferably up to 12 nt, or up to 10 nt or up to 8 nt.

Specifically, the starter ds oligo or ds assembly comprises two overhangs, in particular one overhang at the 5′-end, and one overhang at the 3′-end.

The two overhangs may both be comprised in the leading or the lagging strand in the starter ds oligo or ds assembly, or one overhang may be comprised in the leading strand and the second one in the lagging strand.

Specifically, the starter ds oligo or ds assembly comprises two overhangs, wherein both are 5′-overhangs, one in the leading strand at its 5′-end and one in the lagging strand at its 5′-end.

Specifically, there is no overhang at the 3′-end of a strand if there is an overhang at the 5′-end of the other strand in the starter ds oligo or ds assembly, and vice versa.

Specifically, each overhang of the starter ds oligo or ds assembly comprises or consists of at least any one of 2, 3, 4, 5, 6, 7, 8, 9, 10, 11 or 12 nucleotides e.g., up to 12, 11, 10, 9, 8, 7, 6, 5, 4, 3, or 2 nt. Specifically, the length of the overhang ranges between 2 and 6 nt, preferably about 4 nt (i.e., 4 nt+/−1 nt).

Specifically, each overhang has a length that is not more than half of the starter ds oligo or ds assembly.

Specifically, each overhang of the starter ds oligo or ds assembly comprises or consists of a sticky sequence e.g., a sticky sequence which has a length that is at least any one of 50%, 60%, 70%, 80%, 90% or 100% of the length of the overhang.

Specifically, the sticky sequence is a contiguous nucleotide sequence within the overhang. Specifically, the sticky sequence is positioned within the overhang such that the sticky sequence comprises the terminal nucleotide of the overhang.

Specifically, the sticky sequence of an overhang of the starter ds oligo or ds assembly is at least any one of 2, 3, 4, 5, or 6 nucleotides long, in particular a length that is the same as the sticky terminal sequence of an NA building block, such as an ExtON, to allow hybridization over the full length of the sticky sequences.

According to a specific example, an overhang of the ds assembly consisting of 2 nucleotides comprises a sticky sequence consisting of 2 nt which matches 2 complementary nucleotides of the sticky terminal sequence of an ExtON, to allow hybridization of the matching sequences.

According to a specific example, an overhang of the ds assembly consisting of 3 nucleotides comprises a sticky sequence consisting of 3 nt which matches 3 complementary nucleotides of the sticky terminal sequence of an ExtON, to allow hybridization of the matching sequences.

According to a specific example, an overhang of the ds assembly consisting of 4 nucleotides comprises a sticky sequence consisting of 4 nt which matches 4 complementary nucleotides of the sticky terminal sequence of an ExtON, to allow hybridization of the matching sequences.

According to a specific example, an overhang of the ds assembly consisting of 5 nucleotides comprises a sticky sequence consisting of 5 nt which matches 5 complementary nucleotides of the sticky terminal sequence of an ExtON, to allow hybridization of the matching sequences.

According to a specific example, an overhang of the ds assembly consisting of 6 nucleotides comprises a sticky sequence consisting of 6 nt which matches 6 complementary nucleotides of the sticky terminal sequence of an ExtON, to allow hybridization of the matching sequences.

Specifically, each overhang of the ds assembly comprises a sticky sequence to match the respective sticky terminal sequences of ExtONs.

Specifically, one or two ExtONs can be hybridized to a ds assembly. An ExtON may be hybridized to one side or both sides of a ds assembly.

Specifically, each overhang of the ds assembly is hybridized to an ExtON.

According to a specific aspect, an ExtON is hybridized to only one of the two overhangs of the ds assembly, whereas the other one of the two overhangs is not extended by hybridizing to an ExtON.

According to a specific aspect, an ExtON is hybridized to each of the two overhangs of the ds assembly, e.g., either consecutively or simultaneously. Specifically, two ExtONs are hybridized to two overhangs of the ds assembly.

For example, two ExtONs are used for hybridization to the ds assembly, wherein the sticky sequences of said two ExtONs are matching for hybridization with the two overhangs of the ds assembly, thereby obtaining an extended ds assembly that comprises an ExtON on each side.

In particular, where two ExtONs are used for hybridization to the ds assembly, the sticky sequence of a first ExtON is matching for hybridization with one of the two overhangs of the ds assembly, and the sticky sequence of a second ExtON is matching for hybridization with the other one of the two overhangs of the ds assembly. Specifically, the sticky sequences of the first and second ExtONs differ from each other. Specifically, the sticky sequences of the first and second ExtONs do not comprise a complementary sequence such as to avoid undesired annealing of the two ExtONs to each other.

Specifically, one ExtON can hybridize to the overhang of the leading strand of a ds assembly, thereby obtaining an extension of the lagging strand.

Specifically, one ExtON can hybridize to the overhang of the lagging strand of a ds assembly, thereby obtaining an extension of the leading strand.

Specifically, one ExtON can hybridize to the overhang of the leading strand of a ds assembly and another ExtON to the overhang of the lagging strand, thereby obtaining an extension of both strands.

According to a specific aspect, an ExtON is hybridized to an overhang of the ds assembly through the ExtON's sticky terminal sequence which is sufficiently complementary to the overhang sequence.

Specifically, the sticky terminal sequence can be comprised of the 5′-terminal sequence of the ExtON, and the tail is comprised of or attached to the 3′-terminal sequence of the ExtON.

Alternatively, the sticky terminal sequence can be comprised of the 3′-terminal sequence of the ExtON, and the tail is comprised of or attached to the 5′-terminal sequence of the ExtON.

Specifically, where two ExtONs are used in a method of producing an extended ds assembly as described herein, both ExtONs are characterized by
a. either the sticky terminal sequence of the ExtON is comprised of the 3′-terminal sequence of the ExtON, and the tail is comprised of or attached to the 5′-terminal sequence of the ExtON;
b. or the sticky terminal sequence of the ExtON is comprised of the 5′-terminal sequence of the ExtON, and the tail is comprised of or attached to the 3′-terminal sequence of the ExtON;

According to a specific aspect, an ExtON as used herein comprises a sticky terminal sequence on one side, and an identifiable tail on the other side.

Specifically, the length of the sticky terminal sequence of an ExtON is at least any one of 2, 3, 4, 5, or 6 nt, preferably at least 4 nt or at least 6 nt.

Specifically, a sticky terminal sequence of an ExtON can comprise or consist of at least any one of 2, 3, 4, 5, or 6 nucleotides.

Specifically, the length of the sticky terminal sequence of an ExtON is up to any one of 30, 29, 28, 27, 26, 25, 24, 23, 22, 21, 20, 19, 18, 17, 16, 15, 14, 13, 12, 11, 10, 9, 8, 7, 6, 5, 4, 3, or 2 nt, preferably up to any one of 20 nt, 10 nt 8 nt, 6 nt or 4 nt.

According to a specific aspect, the identifiable tail of the at least one ExtON comprises a unique molecular identifier (UMI), such as an identifiable nucleotide sequence, a barcode, tag, label or molecular moiety. In particular, an ExtON may comprise an identifiable tail which comprises a barcoded nucleotide sequence.

Uniqueness of the identifier can be such that one or both ExtONs used in support of the ligating step c) in the method described herein, comprises only one type of UMI, in particular only one UMI that can be differentiated from other UMIs.

According to a specific aspect, the same UMI is used in a set of ExtONs that is used in the ligating step. Specifically, where two ExtONs are used in a method described herein, both of the ExtONs comprise the same UMI.

According to another specific aspect, different UMIs are used in a set of ExtONs that is used in the ligating step. Specifically, where two ExtONs are used in a method described herein, one of the ExtONs ExtON comprises a UMI that differs from the UMI of the other one of the ExtONs.

In an ExtON set or library, the ExtON library members may contain barcodes specific for each input molecule, or a barcode specific for a subset of ExtONs, or specific for all ExtONs in a library.

Specifically, the identifiable tail or UMI may comprise or consist of a nucleotide sequence of at least 3, 4, 5, or 6 nt e.g., DNA and/or RNA and/or synthetic nucleotides.

Specifically, the ExtON, identifiable tail or UMI comprises or consists of a nucleotide sequence of DNA and RNA nucleotides.

Specifically, the ExtON, identifiable tail or UMI comprises or consists of a nucleotide sequence of DNA, RNA and XNA nucleotides.

Specifically, the ExtON, identifiable tail or UMI comprises synthetic nucleotides such as xenonucleotides (XNA or XNA nt), in particular wherein the UMI comprises one or more XNA nt.

According to a specific aspect, the ExtON, identifiable tail or UMI comprises an XNA nt, wherein the XNA nt comprises or consists of any one of: a nt analog, a modified nt, a non-naturally occurring nt, a synthetic nt, a locked nt (LNA), or a functionalized nt, such as comprising a detectable label e.g., a CLICK-functionalized nucleotide for labeling. Specifically, the XNA is a nucleotide comprising a detectable label which is a fluorophore, a chromaphore or a radioisotope. Specifically, the fluorophore is Cy2, Cy3, Cy5, or an Alexa fluorophore; for example, 488, 568, 555, 647, 708.

According to a specific aspect, the ExtON, the identifiable tail or UMI comprises a chimeric or hybrid nucleotide sequence comprising:
a) a DNA backbone, and at least one RNA nt and/or XNA nt; or
b) an RNA backbone, and at least one DNA nt and/or XNA nt; or
c) an XNA backbone, and at least one DNA nt and/or RNA nt.

According to a specific aspect, the ExtON may comprise a DNA backbone, a sticky sequence, and the first nucleotide following the sticky sequence at the 3′-end is an Uracil (“U”) Specifically, the U is the 5′ terminal nucleotide of the identifiable tail.

Specifically, the identifiable tail or UMI comprises or consists of at least 4 DNA and/or RNA nt. Specifically, such sequence may comprise or consist of a barcode. Specifically, a UMI may be a ss oligo which is at least 8-mer, 9-mer, 10-mer, 11-mer or 12-mer.

Specifically, the UMI may be a sequence of n bases, wherein n is any number between 8 and 30 e.g., 10-30 bases, each base being selected from N (“N”, the IUPAC code for any base). Such UMI can be recognized and bound by a respective sequence that is complementary to the UMI sequence.

Specifically, the UMI may be a poly-N sequence, which can be recognized and bound by a respective sequence of a complementary poly-N sequence.

For example,
a) the UMI may be a poly-T sequence, which can be recognized and bound by a respective poly-A sequence;
b) the UMI may be a poly-A sequence, which can be recognized and bound by a respective poly-T sequence;
c) the UMI may be a poly-C sequence, which can be recognized and bound by a respective poly-G sequence;
d) the UMI may be a poly-G sequence, which can be recognized and bound by a respective poly-C sequence;
e) the UMI may be a poly-U sequence, which can be recognized and bound by a respective poly-A sequence

Specifically, the identifiable tail or UMI may comprise a nucleotide linker sequence that is linking an affinity tag or moiety to the ExtON e.g., a nucleotide linker sequence comprising DNA and/or RNA and/or synthetic nucleobases.

Specifically, an RNA linker is used for binding an affinity tag or moiety. Upon an RNase digest the ds assembly can be separated from the affinity tag.

Specifically, the affinity tag can be any moiety such as an affinity label or ligand e.g., a polyA tail, a biotin moiety, a sterol lipid moiety, a click chemistry moiety-based label, or a combination of any of the foregoing.

Specifically, a sterol lipid moiety can be e.g., cholesterol.

Specifically, a click chemistry moiety can be a click chemistry-based label such as e.g., labels enabling Azide-alkyne cycloadditions (AAC) for labelling, ligation, and cyclization.

Specifically, the identifiable tail or UMI may comprise a target site for a displacement reaction, such as by cleavage and/or degradation e.g., by an enzymatic reaction. In particular, the target site may be a cleavage site specifically recognized by a nuclease. By a respective enzymatic reaction, the ExtON can be cleaved off and/or degraded.

According to specific examples,
a) an ExtON comprising a poly(A) tail, such as e.g., a poly A10-30 tail, can be specifically recognized by a poly(T) mRNA capture sequence;
b) an ExtON comprising a barcode tail, can be specifically recognized by a bait reagent, moiety, or device;
c) an ExtON comprising an affinity ligand tail, can be specifically recognized by an affinity-based receptor e.g., the ExtON can be biotinylated, such that the affinity ligand is biotin, which is specifically recognized by streptavidin used as an affinity-based receptor, which is e.g. immobilized on a solid support such as beads,
d) an ExtON comprising a sterol lipid moiety tail, such as e.g. cholesterol, can be specifically recognized by lipid bilayers and monolayer lipid membranes e.g., of lipid vesicles or immobilized lipid layers;
e) an ExtON comprising a click chemistry moiety tail, such as e.g., labels enabling Azide-alkyne cycloadditions (AAC) for labeling, ligation, and cyclization, such labels can be used to chemically modify the ExtON before removal; as an example, an ExtON can be removed upon its cyclization by click chemistry;
f) an ExtON comprising a tail of RNA nt can be recognized by an RNase, preferably RNAse H. Specifically, a RNase digest can be used to digest, cleave, or degrade the ExtON;
g) an ExtON comprising an RNA part e.g., a tail of RNA nt, can be used to separate the DNA ds assembly from the ExtON upon cleavage by an RNase;
h) the Exton comprising a chimeric or hybrid nucleotide sequence comprising a heterologous nt or nt sequence, such as e.g., comprising:
   i) a DNA backbone, and at least one heterologous nt that is an RNA nt and/or XNA nt; or
   ii) an RNA backbone, and at least one heterologous nt that is a DNA nt and/or XNA nt; or
   iii) an XNA backbone, and at least one heterologous nt that is a DNA nt and/or RNA nt.

Specifically, in an ExtON comprising a DNA backbone, an RNA and/or XNA nt can be recognized for a displacement reaction. Specifically, in an ExtON comprising an RNA backbone, a DNA and/or XNA nt can be recognized for a displacement reaction.

Specifically, the ds assembly obtained upon annealing the source ss oligos and/or hybridizing one or more NA building blocks, can comprise one or two or more than two nicks, which typically depends on the number of NA building blocks comprised in the ds assembly, in particular the number of source ss oligos comprised in the starter ds oligo and/or the type of NA building block that is hybridized to the starter ds oligo to produce the ds assembly, the assembly method and conditions which may result in insufficient ligation of the strands of the NA building blocks within the ds assembly.

A nick may result upon assembly of the ss oligos and/or NA building blocks that are adjacent in a strand of the ds assembly. Specifically, a nick is between the terminal nucleotides of the adjacent ss oligos and/or NA building blocks. As an example, there may be a nick where there is no nucleotide bond between the 5′ terminal nucleotide of one of the adjacent ss oligos and the 3′-terminal nucleotide of the other one of the adjacent ss oligos. According to another example, there may be a nick within a strand upon assembly of two adjacent NA building blocks e.g., where there is no nucleotide bond between the 5′ terminal nucleotide of one of the adjacent NA building blocks and the 3′-terminal nucleotide of the other one of the adjacent NA building blocks.

Following ligation, a nick can be “resolved” or “closed”, e.g., by an enzymatic reaction, creating a nucleotide bond (in particular phosphodiester bond) between the adjacent terminal nucleotides of NA building blocks.

According to a specific example, annealing and hybridizing of 3 ss oligos may produce a ds assembly with two overhangs and one nick in the ds part of the starter ds oligo.

According to a specific example, annealing and hybridizing of 4 ss oligos may produce a ds assembly with two overhangs and two nicks in the ds part of the starter ds oligo.

According to a specific example, annealing and hybridizing of 5 ss oligos may produce a ds assembly with two overhangs and three nicks in the ds part of the starter ds oligo.

According to a specific example, annealing and hybridizing of 6 ss oligos may produce a ds assembly with two overhangs and four nicks in the ds part of the starter ds oligo.

According to a specific aspect, in the annealing step a) of the method described herein, at least 4 ss oligos are annealed and hybridized to produce one ds assembly comprising at least two nicks.

According to a specific aspect, the annealing step a) of the method described herein comprises annealing and hybridizing four ss oligos, by annealing a first and a second pair of ss oligos, wherein each ss oligo pair comprises two ss oligos that comprise a part matching for annealing the ss oligos of the pair, and another part to obtain an overhang of at least 2 nt on each side upon annealing, and wherein one overhang of the first ss oligo pair is matching for hybridization with one overhang of the second ss oligo pair to produce said ds assembly.

According to a specific aspect, in the annealing step a) of the method described herein, where source ss oligos are assembled, assembly reactions can be carried out by only one assembly reaction, or by more than one assembly reactions, such as e.g., simultaneous, parallel, consecutive, or stepwise assembly reactions.

Specifically, in the annealing step a) of the method described herein, all ss oligos are assembled in only one reaction containment, either simultaneously or consecutively, such reaction containment is also referred to as a pool reaction containment.

According to a specific aspect, the annealing step a) of the method described herein is carried out in a pool reaction containment e.g., by a simultaneous or pool assembly reaction, or by consecutive or stepwise assembly reactions.

Specifically, by a pool assembly reaction, assembly of all source ss oligos is carried out in the presence of all source ss oligos.

Specifically, by consecutive assembly reactions, assembly of source ss oligos is carried out stepwise e.g., wherein source ss oligos are assembled in a first assembly reaction, and at least one additional source ss oligo is added to the assembly produced in the first assembly reaction.

Specifically, consecutive reactions can be carried out in the same or different reaction containments.

According to a specific aspect, the annealing step a) where source ss oligos are assembled, can be carried out in more than one reaction containments.

According to a specific example, a first assembly reaction can be carried out in a first reaction containment, to anneal e.g., two ss oligos thereby providing a starter ds oligo as an intermediate reaction product, and a second and further assembly reactions can be carried out in a one or more further reaction containments to anneal and/or hybridize one or more additional NA building blocks, such as ss oligos, ds oligos or ds polynucleotides, to the intermediate reaction product of the first reaction containment.

According to a specific example, a first assembly reaction can be carried out in a first reaction containment, to anneal e.g., two ss oligos thereby providing a ds oligo as a first intermediate reaction product, and a second assembly reaction can be carried out in a second reaction containment, to anneal e.g., two ss oligos thereby providing a ds oligo as a second intermediate reaction product. In a further assembly reaction, the first and second intermediate reaction products are assembled e.g., with or without using one or more additional ss oligos, to produce the ds assembly e.g., in a third reaction containment.

According to another specific example, a first assembly reaction can be carried out in a first reaction containment, to anneal e.g., two ss oligos thereby providing a ds oligo as a first intermediate reaction product, and a second assembly reaction can be carried out in a second reaction containment, to produce e.g., a ds polynucleotide as a second intermediate reaction product. In a further assembly reaction, the first and second intermediate reaction products are assembled e.g., with or without using one or more additional ss oligos, to produce the ds assembly e.g., in a third reaction containment.

Specifically, the hybridizing step b) of the method described herein, where one or more ExtONs are hybridized to the ds assembly, is carried out in only one reaction containment, e.g., by a pool reaction.

Specifically, the hybridization step b) can be carried out in more than one reaction containments such as e.g., in two reaction containments.

For example, a first ExtON is hybridized to a first overhang in a first reaction containment, and a second ExtON is hybridized to a second overhang in a second reaction containment. Such hybridization step b) that is carried out in two reaction containments is typically carried out consecutively e.g., stepwise, thereby obtaining an extended ds assembly that comprises the ds assembly that is extended by the first and second ExtONs.

According to a specific aspect, the annealing step a) and the hybridizing step b) of the method described herein, are carried out in only one reaction containment, either consecutively or simultaneously.

According to a specific aspect, the annealing step a), and the hybridizing step b) of the method described herein, are carried out in more than one reaction containment, either consecutively or simultaneously, e.g., wherein reaction steps are carried out in parallel and/or stepwise.

According to a specific aspect, the ligating step c) of the method described herein follows the hybridization step b), in particular wherein the respective reactions are performed consecutively.

Specifically, the ligating reaction can be carried out in a reaction containment that is also used in the hybridization step, or in a separate reaction containment. In particular the reaction product of the hybridization step can be transferred to another reaction containment to perform the ligation reaction.

According to a specific aspect, the annealing step a), the hybridizing step b), and optionally the ligating step c) of the method described herein, are carried out in one reaction containment, either consecutively or simultaneously, e.g., in a pool reaction.

According to a specific aspect, any one or more of the assembly reactions of the method described herein, in particular any one or more of the annealing, hybridizing and/or ligating reactions, can be carried out in one or more reaction containments of a respective device comprising one or more reaction containments.

A “reaction containment” as referred to herein is understood as a device or part of a device which allows spatial separation of a reaction mixture, in particular providing a reaction site, such as a container, compartment of a multicompartment device, or a spot on the surface of a device.

Specific examples of reaction containments include e.g., reaction tubes, multi-well plates, microfluidic chip devices, capillary devices, or other array devices. Specific reaction containments are array compartments such as spots.

Specifically, a reaction containment or a storage containment is a compartment unit of a device, such as a well, compartmented surface or spot, or any one of a microtiter plate, a microfluidic microplate, a set of capillaries, a microarray or a biochip, preferably a DNA and/or RNA biochip.

Specifically, NA building blocks, such as e.g., ss oligos or ExtONs, are provided and used as described herein, wherein different NA building blocks are provided in separate containments.

Specifically, an array device may be used. Such array device may be any one or more of a microtiter plate, microfluidic microplate, set of capillaries, microarray or a biochip, preferably a DNA or RNA biochip. Said array device may comprise only one, all or any type of the aforementioned containments.

According to a specific aspect, a microfluidics chip can be used in order to pipe droplets containing NA building blocks in such a way as to implement the assembly workflow. Similarly, digital microfluidics e.g., platforms of electro-wetting on dielectric (EWOD) can also be used to have accurate control on the movement of individual droplets, thereby facilitating the implementation of complex hierarchical workflows.

According to a specific aspect, a microtiter plate can be used, which term is understood to refer to well plates, multi-well plates or micro-well plates. These plates are commonly manufactured in a 2:3 rectangular mix with 96, 384, or 1536 wells, although other cavity configurations are available. Some of the other sizes, far less common, available are 6, 24, 3456, and 9600 wells. The wells of the microplate typically hold between tens of nanoliters to several milliliters of liquid.

Capillaries can be any glass capillaries, microfluidic capillaries and autonomous microfluidic capillary systems. Capillary microfluidics are important tools in many different fields. Due to their axisymmetric flow and ability to withstand organic solvents, when compared with their lithographically fabricated polydimethylsiloxane (PDMS) counterparts, glass capillary devices possess advantages for microfluidic applications. In particular, a circular tube is inserted into a square outer flow channel, which greatly simplifies alignment and centering of these devices. These devices can produce small and large droplets, ranging from 10 to multiple hundreds in micrometer size.

NA building blocks may be conveniently transferred from a source such as an array device to reaction containments by automated means e.g., either robotically or via dedicated fluids using, for example, an automated liquid handler, from such compartments into other compartments herein referred to as reaction compartments i.e., from one vessel to another. In order to facilitate time efficient assembly of polynucleotides, hierarchies of reactions and respective vessels may be employed corresponding to frequency of use of NA building block library members. The transfer to a new vessel involves the physical movement of a device that picks one or more molecules of a NA building block from the respective location, or the pneumatic/hydraulic deposition though microfluidics.

According to a specific aspect, NA building blocks can be obtained from a library of NA building blocks, which comprises a diversity of NA building blocks that is provided in separate library containment.

An ExtON set described herein is also referred to as library of NA building blocks such as an ExtON library, which comprises or consists of library members which are each ExtONs with a diversity of sticky terminal sequences.

In a specific embodiment, a library of NA building blocks can be provided within an array device, wherein different library members are contained in separate reaction or storage containments.

Specifically, NA building block library members contained in one reaction or storage containment can be a mixture of different NA building blocks of such sequences that are not capable of annealing to each other, thus, the NA building blocks are contained in the mixture as ss molecules. Specifically, at least two different library members can be contained in one reaction or storage containment of an array device.

According to a specific aspect, transfer of NA building blocks described herein, such as to transfer from one containment to another, e.g., transfer from a library containment to a reaction containment, is done using an automated system e.g., using a liquid handler such as further described herein.

A library of NA building blocks is conveniently used as a source of NA building blocks. NA building blocks can be selected from a library on demand, such as to allow assembling the NA building blocks as described herein.

A library of NA building blocks comprises or consists of library members which are each NA building blocks with a diversity of nucleotide sequences. Specifically, the library of NA building blocks is a library of ss molecules, such as to encompass ss oligos and/or ExtONs.

Specifically, the library members are pre-built, optionally provided in a storage stable form, and provided at defined positions within an array device. Library members can be synthesized and stored in the array device until needed.

Specifically, said library comprises NA building blocks wherein said NA building blocks have been obtained by de novo synthesis, such as by chemical or enzymatic methods of production.

Specifically, NA building blocks can be produced by any suitable prior art method, such as chemical polynucleotide (or oligonucleotide) synthesis methods, including the H-phosphonate, phosphodiester, phosphotriester or phosphite triester synthesis methods, or any of the massively parallel oligonucleotide synthesis methods e.g., microarray or microfluidics-based oligonucleotide synthesis.

Specifically, NA building blocks can be produced by any of the enzymatic polynucleotide (or oligonucleotide) synthesis methods e.g., ssDNA synthesis by DNA polymerase proteins or by reverse transcriptase proteins, which produce hybrid RNA-ssDNA molecules. Specifically, the enzymatic polynucleotide synthesis reaction is performed in vitro.

Specific embodiments refer to NA building blocks which are modified by any one or more of phosphorylation, methylation, biotinylation, or linkage to a fluorophore or quencher.

Specifically, an ExtON set or library described herein may comprise library members which are ExtONs that can be any one or more of the following: unmodified ss; phosphorylated ss; methylated ss; biotinylated ss; or phosphorylated, biotinylated and methylated ss.

Specifically, an ExtON set or library described herein comprises ExtONs which may or may not comprise a 5′-phosphorylation.

Specifically, ExtONs are not phosphorylated and/or comprise a terminal blocking modification e.g., a 5′- or 3′-blocking modification that prevents ligation, preferably a modification that prevents phosphorylation.

Specifically, the ExtON set or library described herein comprises ExtONs which may or may not comprise a fluorophore or quencher.

Specifically, NA building blocks such as e.g., ExtONs, are provided in a storage-stable form, preferably a form which is storage-stable for at least 6 months at room-temperature.

Specifically, an ExtON set or library described herein is storage stable, comprising the ExtONs in storage containments.

Specifically, the library of NA building blocks such as e.g., ExtONs, described herein may comprise NA building blocks in a liquid phase (e.g., an aqueous medium with or without organic solvents such as or alcohol), or dried NA building blocks. NA building blocks can be stored in storage containments in a dry state. Dry-state is, for example, achieved by lyophilization, freeze drying, evaporation, crystallization or the like. The enzymes which catalyze the degradation of nucleic acids are typically active at room temperature in a fluid biomolecule preparation. Dry-state storage inhibits such enzymatic activity because such enzymes are generally inactive upon de-hydration and because the degradative chemical reactions which they catalyze typically entail the addition of water (i.e., hydrolysis) of a protein or nucleic acid molecule, thus producing protein or nucleic acid backbone cleavage. In the dry state, there is little or no water (e.g., less than 5%, 4%, 3%, 2% or 1% (w/w) water) as a chemical reactant to support such enzyme catalysis. Additionally, any non-enzymatic hydrolysis of protein or nucleic acid is similarly inhibited, since water is generally unavailable for such reactions.

According to a specific aspect, the ligation reaction is an enzymatic reaction, preferably wherein the enzyme is a ligase, polymerase, or ribozyme, preferably any one of a T3, T4 or T7 DNA ligase, Taq DNA Ligase, or DNA polymerase.

Preferably T4 DNA ligase, T7 DNA Ligase, T3 DNA Ligase, or engineered respective enzymes are used in the ligation reaction. According to a specific example, the following ligation reaction is used: T4 DNA Ligase, at a concentration of 10 cohesive end units per μL supplemented with 1 mM ATP (Sambrook and Russel, 2014, Chapter 1, Protocol 17).

Following the ligating step c) in the method described herein, the at least one ExtON is typically removed from the ligation product. Specifically, removal of the ExtON(s) is by a displacement reaction.

The displacement reaction may comprise any one or more of dehybridization, cleavage, degradation, destruction, modification (such as e.g., cyclization of the ExtON or other chemical reactions to inactivate or block the sticky end of the ExtON), or isolation steps.

According to a specific example, said at least one ExtON is displaced or separated from the ligation product and/or the ligation product or said at least one ExtON is removed from the ligation reaction mixture. Removing said at least one ExtON can be immediately following the ligation reaction, or after one or more further reaction steps e.g., one or more further assembly steps.

According to a specific aspect, the at least one ExtON can be removed from the extended ds oligo by and/or following dehybridization. Upon dehybridization, the ExtON is no longer extending the ds assembly, but physically separated from the ds assembly, and can be removed from the ds assembly.

Specifically, one or more, in particular all ExtONs used in the extended ds assembly are removed from the ds assembly upon ligation.

According to a specific aspect, the at least one ExtON can be removed by dehybridization and optionally isolating said at least one ExtON or the ligated product, in particular by isolating the target ds polynucleotide.

An ExtON that is separated from the ligation product can be conveniently isolated upon contact with a ligand or binder that recognizes and binds to the ExtON, in particular the identifiable tail of the ExtON and/or the UMI incorporated within the ExtON.

Separated, degraded or modified ExtONs can be removed from the ligated ds assembly by conventional means, such as e.g., enrichment, depletion or washing steps.

According to a specific aspect, the method is characterized by one or more of the following:
a) dehybridization is by increasing the temperature, changing the pH (e.g., low, alkaline pH (<pH 5) or high, acidic pH (>pH 9)), or by chaotropic agents (e.g., guanidine, urea), or other agents interfering with hybridization of nucleic acid molecules such as betaine, a betaine analogue, or organic solvents (e.g., DMSO, formamide);
b) displacement is by enzymatic cleavage or digestion e.g., using an enzyme recognizing a cleavable linker; as an example, RNase such as RNAse H, can be used to digest an RNA part of an ExtON;
c) displacement by chemical modification of the ExtON;
d) isolating said at least one ExtON is by a method employing means or binders that recognize the UMI incorporated in the ExtON;
e) isolating said at least one ExtON is by a method employing means or binders that recognize the ExtON tail e.g., by a non-covalent or covalent bond, hybridization or other affinity reaction, preferably wherein said means employ binders recognizing an affinity tag;
f) isolating said at least one ExtON is by a click chemistry conjugation method;
g) isolating said at least one ExtON is by size-cutoff based magnetic bead-based purification, which removes reactants of size smaller than the ds assembly or ligation product;
h) isolating said target ds polynucleotide is by using an RNAse, preferably RNase H, to digest an RNA-linker region between the DNA-assembly and RNA-terminal affinity moiety;
i) isolating said target ds polynucleotide upon recognizing a heterologous nt or nt sequence in said at least one ExtON comprising a chimeric or hybrid nucleotide sequence, wherein the ExtON comprises:
   i) a DNA backbone, and at least one heterologous nt that is an RNA nt and/or XNA nt; or
   ii) an RNA backbone, and at least one heterologous nt that is a DNA nt and/or XNA nt; or
   iii) an XNA backbone, and at least one heterologous nt that is a DNA nt and/or RNA nt;
j) isolating said target ds polynucleotide by means of separation through electrophoresis, capillary electrophoresis, high-pressure liquid chromatography (HPLC), or any other suitable form of chromatography.

Upon recognizing a heterologous nt or nt sequence in said at least one ExtON comprising a chimeric or hybrid nucleotide sequence, said at least one ExtON can be displaced by a displacement reaction.

According to a specific example, the at least one ExtON is dehybridized and removed by size-cutoff based magnetic bead-based purification.

Binders that recognize and bind the ExtON tail e.g., by a non-covalent or covalent bond, are well-known. Binders may or may not be attached to molecular structures or solid supports, such as beads or spheres, which facilitate physical separation of the ExtON upon binding.

According to a specific aspect, a solid phase such as beads can be used to immobilize ExtONs and/or ExtON binders.

Specifically, beads may comprise any one or more materials selected from a hydrogel, agar, glass, polyacrylamide, polystyrene, or polyethylene.

Typically, molecular entities can be linked to the beads by acrydite linkages, click chemistry, biotin/streptavidin, silane linkage, or amide linkages.

According to a specific example, a binder may specifically recognize and bind an ExtON tail by a non-covalent bond between an affinity tag or moiety and a respective affinity ligand.

According to a specific aspect, the method described herein further comprises producing an assembly of the target ds polynucleotide and one or more NA building blocks such as e.g., DNA building blocks, in one or more further steps, to synthetize a longer ds polynucleotide according to a DNA template.

According to the method provided herein, NA building blocks can be assembled to one or more intermediate ds polynucleotides and ultimately producing a target ds polynucleotide. Specifically, the ligated ds assembly produced by the method described herein can be used as an intermediate ds polynucleotide e.g., used as a NA building block, to produce a longer ds polynucleotide as a target ds polynucleotide

In a method of producing a target ds polynucleotide using a ligated ds assembly as an intermediate NA building block, one or more additional NA building blocks can be assembled according to a defined workflow.

Specifically, the workflow may comprise assembly of one or more ligated ds assemblies wherein at least one of the ds assemblies is produced by the method described herein. Specifically, two or more ds assemblies may be produced by a method described herein e.g., in parallel, such as in one or more assembly tiers.

Specifically, the workflow may comprise assembly of further NA building blocks.

Specifically, one or more ligated ds assemblies as described herein can be used as an intermediate polynucleotide which is a ds NA building block.

According to the method provided herein, NA building blocks are assembled to one or more intermediate polynucleotides and ultimately the target polynucleotide is assembled.

Specifically, the intermediate polynucleotide can be used as a building block to connect further NA building blocks at both ends of said intermediate polynucleotide, such as to assemble at least one NA building block in both directions, in one or more further assembly tiers.

According to a specific aspect, the assembly is a hierarchical assembly in one or more tiers, in particular assembly tiers. Specifically, said assembly is by connecting matching sequences to synthetize a target ds polynucleotide in one or more assembly tiers. Specifically, assembly is by hybridizing complementary nucleotide sequences (or sufficiently complementary sequences) and/or connecting NA building blocks by a ligation reaction, such as comprising enzymatic, chemical, or an adaptor ligation.

Specifically, in an assembly tier, one or more pairs of NA building blocks are assembled. The number of assembly tiers, reaction steps and corresponding reaction products is basically determined by the length of the target polynucleotide. In order to synthesize large target polynucleotides, a series of assembly tiers may be needed for assembly into the target polynucleotide e.g., at least 5, 10, 20, 50, 100, 500, 1.000, 5.000 or more may be necessary. Each tier comprises a series of parallel assembly reactions. Typically, the number of assembly reactions decreases with increasing length of the NA building blocks in a tier.

Specifically, the workflow may provide for the production of ds NA building blocks from respective single stranded (ss) NA building blocks, such as by assembly of ss oligos. Specifically, ds NA building blocks can be produced by assembly of ss NA building blocks before their use in a method to assemble larger polynucleotides.

Specifically, the workflow may provide for the production of ds NA building blocks from respective ds NA building blocks, such as by assembly of ds oligos. Specifically, ds NA building blocks can be produced by assembly of ds NA building blocks before their use in a method to assemble larger polynucleotides.

In a specific aspect of the method described herein, pairs of matching ss oligos are assembled to produce ds NA building blocks. Preferably, said pairs of matching ss oligos are transferred into a reaction containment to produce the respective ds NA building blocks.

Specifically, a ds NA building block has one or two overhangs, which is at one or both of its ends, in particular at the 5′-end and/or at the 3′-end of the ds NA building block.

Specifically, a ds NA building block comprising overhangs on both ends and no blunt end may be used as an intermediate polynucleotide.

Assembling of NA building blocks in both directions results in an extension of the 3′-end and an extension of the 5′-end of said intermediate polynucleotide. Such extensions can be done in parallel and/or in the same assembly tier, or consecutively.

Specifically, the intermediate polynucleotide is assembled with further NA building blocks at its 5′-end and its 3′-end (i.e., at both of its ends) in the same assembly tier, optionally wherein assembly at both ends is carried out simultaneously or consecutively.

Specifically, the intermediate polynucleotide is assembled with further NA building blocks at its 5′-end and its 3′-end (i.e., at both of its ends) in the different assembly tiers, optionally wherein assembly of one of the ends of the intermediate polynucleotide with a NA building block is carried out in one assembly tier, and assembly of the other end of the intermediate polynucleotide with another NA building block is carried out in another assembly tier.

In a specific embodiment, the intermediate polynucleotide is assembled with a NA building block at its 5′-end in a further assembly tier, thereby obtaining a reaction product, which reaction product is assembled with another NA building block at its 3′-end in the same or another further assembly tier.

Assembly of the intermediate polynucleotide with further NA building blocks at both ends can be carried out simultaneously or consecutively.

Where the intermediate polynucleotide is assembled with further NA building blocks at both ends, such polynucleotide is considered as an intermediate that is different from a prefinal polynucleotide.

Typically, in a final step of assembly, a prefinal polynucleotide is used that has been produced in a prefinal assembly step, which is then assembled with a final NA building block in the final step of assembly. Optionally, a final NA building block is used which comprises a blunt end, or an end that is designed to match a site of a nucleotide construct (such as a vector or plasmid) to incorporate the target ds polynucleotide in said nucleotide construct. In such final step of assembly, the prefinal polynucleotide is assembled with another NA building block only at one of its ends.

Specifically, the NA building blocks can be oligonucleotides or polynucleotides e.g., fragments of the ds target polynucleotide.

Specifically, NA building blocks are either double stranded such as ds oligos or ds polynucleotides, in particular ds intermediate polynucleotides or prefinal polynucleotides, or single stranded such as ss oligos or ss polynucleotides.

Specifically, the assembly workflow is a hierarchical workflow.

Specifically, the workflow is determined using an algorithm.

Specifically, said algorithm selects pairs of matching NA building blocks and optionally ss NA building block linkers (such as ss oligo linkers), if necessary, and determines the assembly workflow e.g., not only by a mere sequence partitioning, but also by determining an optimal or near-optimal way to assemble the target ds polynucleotide, avoiding mismatches or undesired reaction products as far as possible.

NA building blocks and assembly workflow can specifically be selected to avoid undesired (incorrect) reactions or reaction products, such as palindromic sequences, runaway reactions and unambiguous assembly.

Specifically, the assembly workflow is automated.

Specifically, the automated workflow employs microfluidic handlers that are capable of transferring serially or in parallel the full or partial contents of one or several compartments into other pre-specified compartments that may or may not be empty.

In certain embodiments of the method described herein, purification of reaction products may be necessary following an assembly step. If there are incorrect reaction products besides the correct reaction products, such incorrect reaction products may be suitably separated from the correct ones e.g., as follows: using gel electrophoresis to detect oligonucleotides or polynucleotides of a certain size and excising and purifying bands of the gel corresponding to the size of the desired reaction product. Specifically, correct reaction products can be detected by incorporation of tags or labels into the sequence. Specifically, oligos or polynucleotides may be captured using biotinylated oligonucleotide adapters capable of hybridizing with an overhang of a NA building block wherein, said adapters are fixed to the substrate and coated with streptavidin. Non-captured incorrect products are eliminated by washing and subsequently, the correct products are released from the adapters by increasing the temperature. Specifically, further separation methods well-known in the art may be applied. Specifically, such methods may involve chromatographic or affinity separation methods.

The invention further refers to a set of extender ss oligo (ExtON).

According to a specific aspect, an ExtON set described herein is composed of at least two different ExtONs which differ at least in the sticky sequences. Specifically, the set may comprise at least 2, 3, 4, 5, 6, 7, 8, 9, 10, 20, 30, 40, 50, 60, 70, 80, 90, 100, 150, 200, 250, 500, or 1000 different ExtONs.

Specifically, a set of ExtONs is provided, wherein each ExtON comprises a sticky sequence composed of a terminal sequence of at least 2 nt on one side, and an identifiable tail on the other side, wherein the identifiable tail comprises a unique molecular identifier (UMI) that is common to all ExtONs in the set, which set comprises a variety of ExtONs to cover a diversity of said sticky sequences.

Specifically, each ExtON of the ExtON set comprises the same UMI and/or the same identifiable tail.

Specifically, the ExtON set is provided in a library of ExtON.

Specifically, the ExtON set is a set that is obtained from an ExtON library.

According to a specific aspect, in the ExtON set the variety of ExtONs is provided in one or more separate containments.

Specifically, a library is provided comprising ExtON library members which differentiate from each other in the sticky sequences. Specifically, library members are provided in separate containments, so to allow obtaining specific ExtONs as needed for carrying out the method described herein.

Specifically, the variety of ExtONs covers a diversity of at least 10 different sticky sequences e.g., at least any one of 10, 20, 30, 40, 50, 60, 70, 80, 90, 100, 150, 200, 250, 500, or 1000 different sticky sequences.

For example, the variety of ExtONs wherein each ExtON comprises a different sticky sequence that consists of a 4-mer of nucleotides, theoretically covers 256 different sticky sequences. A respective ExtON library may comprise 256 different library members, each comprising a different sticky sequence.

Specifically, ExtONs provided in an ExtON set are characterized as further described herein. In particular, each ExtON in a set of ExtONs is characterized by one or more of the ExtON features described herein, in particular features of the ExtON's components, such as the sticky sequence, the UMI and/or the identifiable tail.

Specifically, in an ExtON set, each ExtON is characterized by one or more of the following:
a) the sticky sequence of the ExtON consists of at least 2 nt, preferably at least 4 nt;
b) the sticky sequence of the ExtON is comprised of the 5′-terminal sequence of the ExtON, and the tail is comprised of or attached to the 3′-terminal sequence of the ExtON;
c) the sticky sequence of the ExtON is not phosphorylated and/or comprises a 5′ blocking modification that prevents ligation, preferably a modification that prevents phosphorylation;
d) the ExtON consists of an oligonucleotide of at least 8 nt;
e) the ExtON consists of an oligonucleotide with a length of up to 30 nt, preferably up to 20 nt.

### FIGURES

FIG. 1 List of Sequences of single stranded oligonucleotides.

FIG. 2 ExtONs are inserted in the assembly process to allow ligations of short fragments. In the in-silico process used to partition the sequence, the correct ExtONs are selected for each fragment. (A) ExtONs enabling ligation of short fragments obtained from partitioning of a sequence of interest (SOI) by hybridizing one of the starter ds oligo overhangs with ds oligo, where the ligation is enabled through an ExtON. (B) ExtON enabling ligation of starter ds oligo with a single ss oligo to one of the overhangs. (C) ExtON enabling ligation of starter ds oligo with a ds polynucleotide.

FIG. 3 PAGE results of assemblies without (A) and with (B) ExtONs. The presence of a clear band around the target length of 16 bp only in figure B shows the necessity of this method.

FIG. 4 Result of 4 consecutive, tiered ligation reactions, with starting oligo size of 8 nt and final target molecule of 128 bp, before (A) and after (B) bead enrichment.

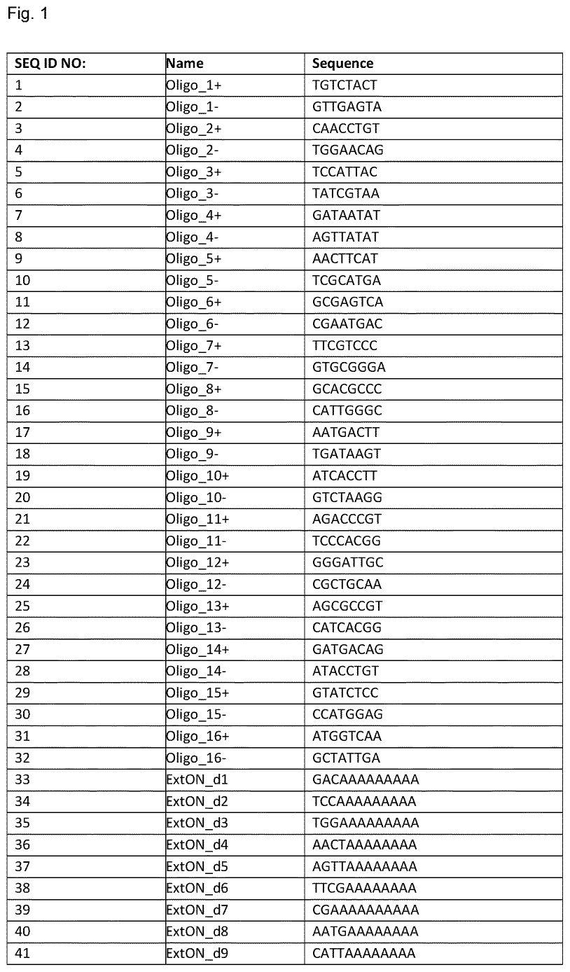

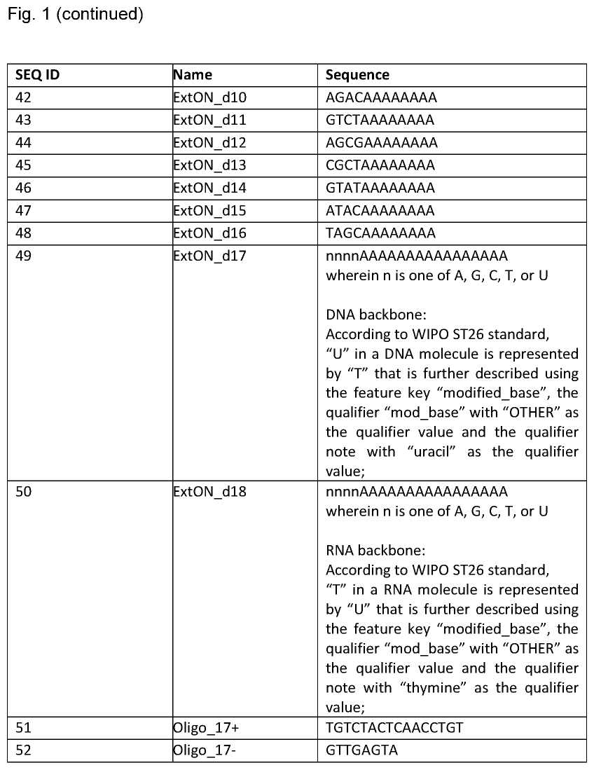

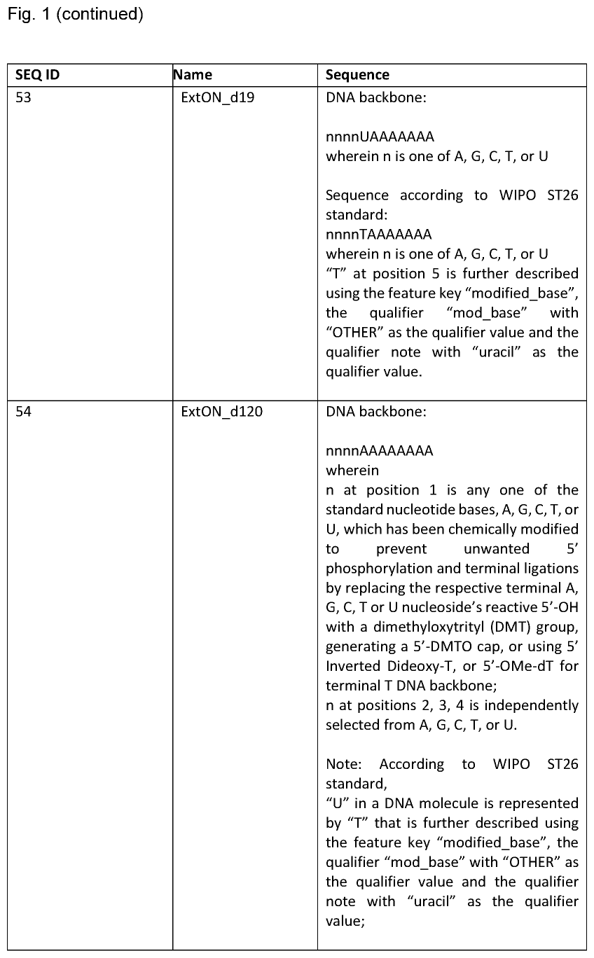

### DETAILED DESCRIPTION

Specific terms as used throughout the specification have the following meaning.

As used herein, the terms “a”, “an” and “the” are used herein to refer to one or more than one i.e., to at least one. The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “assembly” or “assemble” is herein understood as the association or complex comprising a number of at least two ss oligos and/or ds oligos and/or ds polynucleotides with matching sequences forming a construct comprising a ds nucleotide sequence that is composed of the matching sequences. Elements of an assembly, in particular ss oligos and/or ds oligos and/or ds polynucleotides, can be assembled by annealing and/or hybridizing and/or ligation reactions. An assembly may or may not comprise one or more nicks. Nicks can be resolved (i.e., closed) in an assembly by a ligation reaction, such as e.g., by a ligase or polymerase enzyme e.g., to produce an assembly without any nick.

An element of an assembly, in particular a ss oligo and/or ds oligo and/or ds polynucleotide, as used for an assembly, is herein also referred to as a building block e.g., a nucleic acid (NA) building block such as a DNA building block, or hybrid na building block comprising DNA and/or RNA and/or XNA. NA building blocks may be single stranded or double stranded oligonucleotides or polynucleotides as described herein. Upon assembly, e.g., in one or multiple tiers, they can form the target polynucleotide. Therefore, a NA building block can be considered as a subunit of a target polynucleotide comprising a nucleotide sequence of interest (SOI).

For direct assembly, NA building blocks can be joined together by their single stranded parts or overlaps (i.e., the overlapping parts or overhangs). Upon aligning two NA building blocks with an overlap, a continuous sequence can be formed (with or without a nick) which has a length that is the length of both individual NA building blocks taken together. Consequently, a continuous sequence is obtained which comprises a segment that originates from the aligned NA building blocks.

For indirect assembly, a target ds polynucleotide or any intermediates thereof can be formed upon assembly of ss or ds NA building blocks and joining them through one or more ss linker(s). For example, two ss NA building blocks, each of e.g., 10 bases length, may be joined by a ss oligo linker of e.g., of 6 bases length, such that 3 bases of the 3′ terminal end of the first ss NA building block align with the 3 bases of the 5′ end of the ss linker and that 3 bases of the 5′ end of the second ss NA building block align with the 3 bases of the 3′ end of the ss linker.

Single stranded oligo linkers having a sequence complementary to the combined overhangs may connect adjacent NA building blocks in the target polynucleotide. Linkers may e.g., consist of at least 6 bases to connect two adjacent NA building blocks, each with an at least 3 base long overhang, one on the 3′ end and the other on the 5′ end, respectively.

Each assembly will result in an extension of one or both ends of an NA building block, such as an extension of the 5′-end and/or the 3′-end of the NA building bock.

Multiple parallel assembly reactions can be carried out when synthetizing a target polynucleotide. Specifically, an assembly tier is characterized by at least two assembly reactions in parallel, in particular wherein the respective reaction products will be further reacted in one or more further steps. Specifically, one or more parallel assembly reactions can be performed in separate reaction containments during one assembly period (“tier” or “round”), before one or more further assembly reactions are performed in any of the following tiers, using assembly product(s) of any of the previous tiers.

Multiple-tier assembly is herein understood as a multi-part, modular assembly method. Contrary to “one-pot” type of assembly such as in a pool reaction, it particularly refers to an assembly in more than one tier e.g., a hierarchical assembly, where the component DNA pieces are assembled in. Multi-tier DNA assembly methods can be performed with methods that combine or include e.g., Golden Gate assembly, BASIC, BioBrick assembly (and variants such as BglBrick) and Gateway cloning. Bespoke multi-tier DNA assembly methods can also include Gibson Assembly, AQUA cloning, Twin Primer Assembly, ligase cycling reaction, SLIC, SLiCE, overlap extension PCR and CPEC.

To compare, single-tier assembly is understood to assemble a polynucleotide in one step, e.g., using pool reactions or “one-pot” assembly reactions.

An assembly of a number of ss oligos can be used to produce a ds assembly. The ds assembly is typically composed of a number of ss oligos and optionally one or more additional NA building blocks which are ss oligos, ds oligos, or ds polynucleotides, with matching sequences. A ds assembly may or may not comprise one or two or more nicks. A ds assembly typically has one or two overhangs and can be used as an intermediate to produce a longer target ds oligo or polynucleotide through assembly with one or more further NA building blocks in one or more further assembly tiers.

For example, a ds assembly can be composed of at least 2, 3, 4, 5, or 6 ss oligos.

According to another example, a ds assembly can be composed of at least 2, 3, or 4, ss oligos, and one or two ds NA building blocks, such as selected from ds oligos and ds polynucleotides.

An intermediate polynucleotide is typically understood as a product of an assembly of at least two NA building blocks. For example, a pair of matching NA building blocks is transferred into a reaction containment and the matching NA building blocks are assembled thereby forming a new NA building block that is double stranded and considered as an intermediate because used in a further assembly step. Specifically, said ds NA building block may comprise at least one overhang. Such overhang allows further assembly with another matching NA building block in the direction of the overhang. Where an intermediate comprises two overhangs i.e., on both of its ends, such intermediate is designed to assemble with further NA building blocks in both directions.

The term “assembly workflow” or simply “workflow” as used herein refers to the pre-defined sequence of assembly of ds NA building blocks to the target ds polynucleotide. The workflow is typically optimized to allow efficient synthesis of the target polynucleotide, aiming to avoid mismatches of ds NA building blocks which would lead to a polynucleotide comprising an incorrect sequence.

The workflow is specifically designed to avoid mismatches or reaction products which cannot be used for assembly to produce the target ds polynucleotide. If there are partial constructs that can anneal in alternative ways, a runaway i.e., an uncontrolled polymerization reaction, can occur. To avoid combinations of pairs of matching ds NA building blocks that would result in unwanted constructs or runaway reactions, the pairs of matching ds NA building blocks are assembled in a predetermined sequence of assembly steps i.e., a specific workflow. Preferably, said specific workflow is not linear but hierarchical i.e., following an algorithm that provides for intermediate reaction products which are defined non-consecutive parts of the target ds polynucleotide conveniently produced avoiding undesired reaction products to the extent possible, before such intermediate reaction products are further assembled into further intermediate reaction products or into the target ds polynucleotide sequence.

In a linear workflow, the polynucleotide is assembled in a linear fashion starting at the 3′ end of the leading strand, and adding the next oligo to link the 3′ end of the leading strand with the 5′ end of the next oligo. For example, oligo B is ligated to oligo A, oligo C is ligated to oligo B, oligo D is ligated to oligo C and so forth. This assembly may be achieved simultaneously by adding all oligos to the reaction containment at the same time (such as in a pool assembly), or the polynucleotide is extended progressively by successively adding oligos A, B, C, D and so forth to the reaction containment.

In a hierarchical workflow, the polynucleotide is typically assembled in at least two tiers. To generate a target polynucleotide A-B-C-D, in the first tier, oligo B is ligated to oligo A, oligo D is ligated to oligo C, producing the reaction products A-B and C-D. In the second tier, the reaction product A-B is ligated to C-D, thereby producing the target polynucleotide A-B-C-D.

The process of determining the assembly workflow can be carried out by an algorithm. Candidate divisions of the sequence of the template can be systematically examined to find the optimal number and length of the ds NA building blocks, and optimal assembly sequence of subsets for DNA synthesis. Initially, the entire target sequence can be taken as a single subset, after which smaller and smaller subsets can be formed with increasing numbers of candidate ds NA building blocks in decreasing size until a partitioning is found that fulfills the subset criteria.

Specifically, an assembly of subsets of NA building blocks yields intermediate reaction products, also called intermediates, and assembly of intermediate reaction products ultimately yields the target ds polynucleotide. Preferably, additional criteria to those listed above may be used for selecting subsets of NA building blocks. Such additional criteria include, but are not limited to, minimization of the size of the subset of NA building blocks employed in any single ligation reaction (for example to avoid mismatch ligations), minimizing the difference in annealing temperature of members of a subset of NA building block precursors, minimizing the difference in annealing temperatures of the overhangs of different ds subunits, whether to employ frame-shifting adaptors or ss oligo linkers and whether to minimize the degree of cross-hybridization among the hybrid forming portions of different NA building blocks that make up a subset.

The term “algorithm” as used herein refers to a self-contained sequence of actions to be performed. An algorithm is an effective method that can be expressed within a finite amount of space and time and in a well-defined formal language for calculating a function. Starting from an initial state and initial input the instructions describe a computation that, when executed, proceeds through a finite number of well-defined successive states, eventually producing “output” and terminating at a final ending state. The transition from one state to the next is necessarily deterministic.

The term “annealing” as used herein generally refers to a reaction in which at least two ss oligo or ss polynucleotides react to form a complex of nucleotide strands, by applying annealing conditions. The complex may comprise two strands forming a duplex structure, three or more strands forming a multi stranded complex, a single self-hybridizing strand, or any combination of these. The complex can be stabilized via hydrogen bonding between the bases of the nucleotide residues of either strand. Specifically, annealing is understood to refer to a ss oligonucleotide or ss polynucleotide, of which the monomers are capable of specifically binding to other monomers by way of a regular pattern of monomer-to-monomer interactions, such as Watson-Crick type of base pairing, base stacking, Hoogsteen or reverse Hoogsteen types of base pairing, wobble base pairing, or the like.

Typically, the term “annealing” is used for an assembly of ss NA building blocks (such as ss oligos or ss polynucleotides).

The term “hybridization” refers to the formation of a series of “canonicar” hydrogen-bonded base pairs between a contiguous sequence of nucleotides of one ss region (or ss strand) and a matching sequence of nucleotides of another ss region (or ss strand), such that A is paired with U or T and C is paired with G. Hydrogen bonding stabilizes the complex of two ss oligo or polynucleotides.

Typically, the term “hybridizing” is used for an assembly of ds NA building blocks (such as ds oligos or ds polynucleotides) comprising matching ss regions (in particular matching overlaps), although the terms “hybridize,” “anneal,” and “pair” are used interchangeably to describe this reaction, and so too they are used interchangeably herein. Hybridization may proceed between two single-stranded DNA molecules, two single-stranded RNA molecules, or between single-strands of DNA and RNA, to form a double-stranded nucleic acid complex.

Specifically, hybridizing provides ds NA building blocks and their assembly to produce intermediates or target ds polynucleotides. Assembly of ds NA building blocks can be performed by selecting those NA building blocks with matching overhangs, such as to allow hydrogen-bond formation between the matching sequences of the overhangs.

The hybridization efficiency between two complementary sequences or sufficiently complementary sequences depends on the operating conditions that are used, and in particular the stringency. The stringency may be understood to denote the degree of homology; the higher the stringency, the higher percent homology between the sequences. The stringency may be defined in particular by the base composition of the two nucleic sequences, and/or by the degree of mismatching between these two nucleic sequences. By varying the conditions e.g., salt concentration and temperature, a given nucleic acid sequence may be allowed to hybridize only with its exact complement (high stringency) or with any somewhat related sequences (low stringency). Increasing the temperature or decreasing the salt concentration may tend to increase the selectivity of a ligation reaction.

Preferably, hybridization is performed under stringent conditions to ensure that only matching NA building blocks hybridize. Typically, stringency is controlled by adjusting the temperature at which hybridization occurs while holding salt concentration at some constant value e.g., at about (+/−20%) 100 mM NaCl, or the equivalent salt concentration of other salts. Other factors can be relevant, such as the particular sequence or length of the matching pair of NA building blocks.

The term “dehybridization” is herein understood as breaking a series of “canonical” hydrogen-bonded base pairs of a ds NA building block into the respective ss regions at certain conditions, which conditions prevent re-hybridization of the ss regions despite of being matching ss sequences. Dehybridization is herein particularly understood as separation or “detaching” of a hybridized double stranded region into the respective single stranded region. Separation can be at least 50%, 60%, 70%, 80%, more 90%, 95%, 96%, 97%, 98%, 99%, or 100% of a sequence.

Dehybridization can be achieved e.g., by increasing the temperature, increasing the pH, by chemical reagents, or electrophoretic force.

Specifically, dehybridization can mean the separation of an ExtON from a ds NA building block, such as a ds assembly that had been extended by an ExtON. Typically, an ExtON is used to facilitate a ligation process step to close any nicks. Upon ligation, the ExtON can be dehybridized from the ligation product, separated and disposed.

Specifically, the dehybridization of an ExtON from a ligation product provides the ligation product comprising an overhang with a sticky sequence that had been hybridized with the ExtON sticky sequence prior to such dehybridization. Specifically, dehybridization of one or two ExtONs from an extended ds assembly is done after closing any nicks within the ds assembly. Specifically, a ds assembly, such as a ds oligo or polynucleotide, is produced in a first assembly tier. The ds assembly comprising one or two ExtONs to facilitate a ligation reaction may be used to produce longer target ds polynucleotides by further assembly reactions. Before carrying out a further assembly of NA building blocks in a further assembly tier, said one or two ExtONs are typically displaced e.g., dehybridized and removed, from the ds assembly.

The term “library” as used herein shall refer to a collection of library members which are NA building blocks (e.g., an oligonucleotide or polynucleotide library). Library members can be ss NA building blocks or ds NA building blocks. A library typically contains library members which are diverse. The library described herein comprises library members suitably composed of NA building blocks of varying lengths and different sequences.

According to a specific example, the library comprises a set of oligos each with a different sticky sequence, to cover a diversity of sticky sequences.

The library provided herein specifically comprises or consists of a set of ExtONs wherein each ExtON is characterized by a common identifiable feature, such as an identifiable tail. The ExtON set specifically comprises a variety of ExtONs to cover a diversity of sticky sequences. According to a specific example, the diversity covers all possible combinations of nucleotides within a sticky sequence. According to another specific example, the diversity covers only a selection of combinations of nucleotides within a sticky sequence, which selection can be conveniently used in the production of a ds assembly extension.

The library preferably comprises ExtONs which are artificially or chemically synthesized, or chemically modified (e.g., including peptidyl nucleic acids or phosphorothioate bond) ss oligos which are synthesized by suitable methods well-known in the art. The ss oligos comprised in the library can also be generated by enzymatic digestion of naturally occurring DNAs.

The library described herein may comprise a number of ExtONs as library members, each with a different sticky sequence, depending on the length of the sticky sequence. Specifically, the library described herein comprises a diversity of library members, wherein each of the library members has a different nucleotide sequence.

Specifically, said diversity means, that different library members differ in at least one base or base pair. One library member may actually encompass multiple copies of the respective ExtON, which copies consist of the same sequence. Such multiple copies of a library member are specifically contained in only one library containment.

The library described herein specifically comprises or consists of ExtONs which are preferably purified, may comprise modifications and are preferably kept at a standard concentration and volume in an appropriate buffer, solvent and/or excipient, e.g., ready-to-use for assembling, or be provided in a storage-stable form, such as in the dry state.

Specifically, the library members are contained in the library in a storage-stable form, such as in solution or in a dry state.

Specifically, any of the following buffer and/or excipients may be used to keep the NA building blocks in solution: Tris Buffer, T.E. Buffer (Tris-EDTA Buffer) or Nuclease Free Water. Specifically, library members may be kept in Tris Buffer, wherein said Tris Buffer is provided at a concentration of about 10 mM (+/−1 mM or 2 mM). Specifically, library members may be kept in T.E. Buffer. Specifically, said T.E. Buffer is at least composed of Tris, at a concentration of about 10 mM (+/−1 mM or 2 mM), and EDTA, at a concentration of any one of 0.1, 0.2, 0.3, 0.4, 0.5, 0.6, 0.7, 0.8, 0.9 or 1.0 mM. Specifically, Nuclease Free Water, is water which has been de-ionized, filtered and autoclaved and is essentially free of contaminating non-specific endonuclease, exonuclease and RNase activity.

Specifically, any of the following solvents may be used to provide a solution of ExtONs: ethanol, methanol, propanol, formamide, pyridine, or dimethyl sulfoxide, acetonitrile, dimethylformamide, formamide, tetrahydrofuran, MDSO, DMF, glycerol or ionic solvents, or mixtures of any of the foregoing e.g., in varying concentrations.

Specifically, all library members are kept in a compartmented array device.

In a specific embodiment, a library of ExtONs is provided within an array device, wherein library members are contained in separate library containments.

Specifically, said array device is any of a microtiter plate, a microfluidic microplate, a set of capillaries, a microarray or a biochip, preferably a DNA and/or RNA biochip. Said array device may comprise only one, all or any type of the aforementioned containments.

Specifically, more than one different library members may be contained in only one library containment.

Specifically, said different library members contained in one library containment can be a mixture of ExtONs of such sequences that are not capable of annealing to each other, thus, the ExtONs are contained in the mixture as ss molecules.

The term “library diversity” or “diversity” as used herein, refers to a degree of versatility characterizing the library provided herein. Specifically, said diversity comprises ExtONs of different lengths and different sequences.

Specifically, the diversity of an ExtON library means that a variety of library members differ in at least one base or base pair. One library member may actually encompass multiple copies of ExtONs of the same sequence. Such multiple copies of a library member are specifically contained in only one library containment.

For example, the library may comprise all possible sequence variations of 4 nucleobase long sticky sequences, which are 254 different sticky sequences of nucleobases length.

Each library member may be individually characterized and marked by a selectable marker or a DNA sequence tag or barcode, to facilitate the selection of a library member in the library or the identification of a library member in the library. Alternatively, the genetic mutation may be determined directly by a suitable determination method e.g., high-throughput sequencing, capillary sequencing or employing specific probes hybridizing with a predefined sequence, to select the corresponding oligonucleotide.

It may be desirable to place each of the diverse library members in separate containers, to obtain a library of ExtONs in separate containers. According to a specific embodiment, the library is provided in an array e.g., a DNA biochip, wherein the array comprises a series of containments or spots on a solid carrier.

As used herein, the term “ligation” is intended to mean the process during which two nucleotide strands are joined by a covalent bond, such that the 5′-end of a first strand is joined to the 3′-end of a second strand. For example, two ds NA building blocks can be connected by a covalent bond under appropriate conditions. Specifically, the 5′-end and 3′-end of the leading strand of a NA building block can be connected to the 3′-end and 5′-end of the leading strand of another ds NA building block, respectively. Specifically, by ligating two ds NA building blocks, a continuous sequence is formed without nicks. Ligated ds NA building blocks typically have a length that is the length of both individual ds NA building blocks taken together.

Ligation products can be formed from both double stranded nucleic acids and single stranded nucleic acids. Double-stranded nucleic acids can be ligated by “sticky end” ligation or “blunt end” ligation. In sticky end ligation, staggered ends comprising terminal overhangs can hybridize to a ligation partner. In blunt end ligation, terminal overhangs are not present and successful ligation depends on transient associations of 5′-ends and 3′-ends. The ligation of two ds NA building blocks with blunt ends is also understood as an end-to-end ligation.

The ligation of two ds NA building blocks with sticky ends is also understood as a side-to-side ligation, wherein the 5′-end and 3-end of the ss nucleotide sequence which is comprised as a leading strand defines the 5′-end and 3′-end of the ds NA building block, respectively.

Blunt end ligations in general are less efficient than sticky end ligations, and various optimizations, such as adjusting concentrations, incubation times, and temperatures, can be applied to improve efficiencies.

The ligation reaction to produce a ds ligation product is typically performed by hybridizing nucleic acid molecules which are sufficiently complementary to each other, and by an enzymatic reaction to resolve any nicks.

In particular, one or more nicks in the double stranded region of hybridized strands can be closed by a ligation reaction.

The ligation efficiency between two ds NA building blocks depends on the nucleotide length and the hybridization efficiency.

The ligation reaction can be performed by an enzyme, specifically a DNA ligase enzyme. A DNA ligase catalyzes the formation of covalent phosphodiester linkages, which permanently join the nucleotides together. Non-limiting examples of enzymes that can be used for ligation reactions are ATP-dependent double-stranded polynucleotide ligases, NAD+ dependent DNA or RNA ligases, and single-strand polynucleotide ligases. Non-limiting examples of ligases are Escherichia coli DNA ligase, Thermus filiformis DNA ligase, Thermus thermophilus DNA ligase, Thermus scotoductus DNA ligase (I and II), CircLigase™ (Epicentre; Madison, WI), T3 DNA ligase, T4 DNA ligase, T4 RNA ligase, T7 DNA ligase, Taq ligase, Ampligase (Epicentre® Technologies Corp.), VanC-type ligase, 9° N DNA Ligase, Tsp DNA ligase, DNA ligase I, DNA ligase III, DNA ligase IV, Sso7-T3 DNA ligase, Sso7-T4 DNA ligase, Sso7-T7 DNA ligase, Sso7-Taq DNA ligase, Sso7-E. coli DNA ligase, Sso7-Amp ligase DNA ligase, and thermostable ligases. Ligase enzymes may be wild-type, mutant isoforms, and genetically engineered variants. Ligation reactions can contain a buffer component, small molecule ligation enhancers, and other reaction components.

Assembly of ds NA building blocks into intermediate polynucleotides or into the target polynucleotide may be carried out using suitable ligation buffer solutions. The ligation buffer solution is e.g., an aqueous solution, typically in a nuclease-free environment, at a pH that ensures the selected ligase will be active; typically, this is a pH of between about 7-9. Preferably, the pH is maintained by Tris-HCl at a concentration of between about 5 mM to 50 mM. The ligation buffer solution may include one or more nuclease inhibitors, usually calcium ion chelators, such as EDTA. Typically, EDTA is included at a concentration of between about 0.1 to 10 mM. The ligation buffer solution includes whatever cofactors are required for the selected ligase to be active. Usually, this is a divalent magnesium ion at a concentration of between about 0.2 mM to 20 mM, typically provided as a chloride salt. For T4 DNA ligase, ATP is required as a cofactor. The ligase buffer solution may also include a reducing agent, such as dithiothreitol (DTT) or dithioerythritol (DTE), typically at a concentration of between about 0.1 mM to about 10 mM. Optionally, the ligase buffer may contain agents to reduce nonspecific binding of the oligonucleotides and polynucleotides. Exemplary agents include salmon sperm DNA, herring sperm DNA, serum albumin, Denhardt's solution, and the like. Preferably, ligation conditions are adjusted so that ligation will occur if matching NA building blocks form perfectly matched duplexes with the bases of a contiguous complementary region. However, it is understood that it may be advantageous to permit non-pairing nucleotides on the 5′-end of one NA building block of a matching pair of NA building blocks and the 3′-end of the other one of the matching pair of NA building blocks to aid in detection or to reduce blunt-end ligation. Important parameters in the ligation reaction include temperature, salt concentration, presence or absence and concentration of denaturants such as formamide, concentration of the pair of NA building blocks and type of ligase employed.

Preferably, ligation is performed under stringent conditions. Typically, stringency is controlled by adjusting the temperature at which the ligation reaction occurs. The temperature will depend on the heat lability of a selected ligase.

Ligation may be followed by one or more amplification reactions. In some embodiments, the ligation products, or target polynucleotides are isolated or enriched prior to amplification.

Isolation can be achieved by various suitable purification methods including affinity purification and gel electrophoresis. For example, ligation products, or target polynucleotides can be isolated by binding of a selective binding agent immobilized on a support to a tag attached to the capture probe. The support can then be used to separate or isolate the capture probe and any polynucleotide hybridized to the capture probe from the other contents of the sample reaction volume. The isolated polynucleotides can then be used for amplification and further sample preparation steps. In some embodiments, the capture probe is degraded or selectively removed prior to amplification of the circular target polynucleotides. Amplification of reaction products, or target polynucleotides can be achieved by various suitable amplification methods known to those skilled in the art.

The term “matching” as used herein refers to the homology or stringency of ss nucleotide sequences. Two ss nucleotide sequences are understood as being matching, if they are sufficiently complementary to each other e.g., fully complementary to each other, or comprising regions that are complementary or at least partially complementary to each other, such as to allow annealing and/or hybridizing of the respective nucleotide sequences. Sufficiently complementary nucleotide sequences can be at least 50%, 60%, 70%, 80%, 90%, 95%, 96%, 97%, 98%, 99%, or 100% complementary to each other.

Specifically, a matching sequence can refer to a pair of complementary ss nucleotide sequences making up a ds NA building block or to a pair of overhangs connecting two ds NA building blocks.

According to a specific example, two, three, four, five or six ss NA building blocks may comprise matching sequences to allow their annealing or hybridization and assembly of the ss NA building blocks, such as to produce a ds assembly e.g., a ds oligonucleotide or ds polynucleotide, that is composed of the ss NA building blocks.

Specifically, hybridizing of fully complementary ss oligos leads to ds oligos with blunt ends and hybridizing of partially complementary ss oligos leads to ds oligos with overhangs.

Specifically, a ds NA building block may comprise an overhang at one end and a blunt end at the other end.

Specifically, a ds NA building block may comprise an overhang at both ends.

According to a specific example, two overhangs of a ds NA building block may comprise matching sequences to allow their hybridization and assembly of two further NA building blocks.

A specific matching sequence can be the sticky sequence of a ss oligo such as an ExtON, which is sufficiently complementary to an overhang of a ds oligo.

The term “nick” as used herein refers to a break or discontinuity in a continuous nucleotide sequence resulting from two unlinked nucleotides that are adjacent in the continuous nucleotide sequence. Typically, a nick is a single-strand break in a double stranded oligo or polynucleotide. For example, a nick is on only one of the strands of a double stranded oligo or polynucleotide, while the other strand does not have a nick at the corresponding position, thereby avoiding a double-strand break.

A ds assembly, such as a ds oligo or polynucleotide, may comprise one or two or more nicks. Specifically, a nick can refer to one or more discontinuities in the sequence of assembled ds NA building blocks, where there is no phosphodiester bond between adjacent nucleotides of one strand. Specifically, the adjacent nucleotides can be the former 5′ and 3′ terminal nucleotides of two ss or ds NA building blocks in an assembly comprising the two NA building blocks.

Nicks can be incorporated into a ds NA building block or assembly of NA building blocks by a site-specific nicking endonuclease. Yet, as further described herein, a nick can be produced upon assembly of two NA building blocks without respective ligation between the ends of the two building blocks.

For example, if two ds NA building blocks are assembled, the assembly may comprise a nick between the terminal nucleotides of the NA building blocks which are adjacent in one of the strands. If both strands comprise a nick, either nick is still a single-strand break, because the other strand is unbroken between the nucleotides at the corresponding positions of the other strand.

According to specific examples, three or more NA building blocks are assembled, and the assembly may comprise one or more nicks because of unligated terminal nucleotides of adjacent NA building blocks within the assembly. For example, three, four or more ss oligos can be assembled to form a ds assembly such as a ds oligo or ds polynucleotide. The assembly may comprise 1, 2, or more nicks because of unligated terminal nucleotides of adjacent ss oligos and/or NA building blocks within the assembly.

To this end, the term “adjacent” nucleotides within a continuous sequence is herein understood as nucleotides at consecutive positions or contiguous within the continuous sequence. Within a ds assembly of NA building blocks, two assembled NA building blocks are considered to be adjacent, where the nucleotides of the leading and the lagging strands of both NA building blocks form a respective continuous sequence (with or without any nick), thus, a continuous sequence of the leading strand of the assembly, and a continuous sequence of the lagging strand of the assembly. Specifically, the 5′-terminal nucleotide of one NA building block is at an immediately consecutive position or contiguous with the 3′-terminal nucleotide of another NA building block within the continuous sequence.

Nicked ds NA building blocks or nicked ds assemblies of NA building blocks can be subject to ligation, to ligate the break and thereby resolve or close the nick. Ligation of a nick in double stranded DNA or DNA/RNA hybrid is typically specific to the fully complementary sequences at opposite strands around the nick site.

The term “oligonucleotide” (oligo) as used herein refers to a linear polymer of nucleotide monomers, also referred to as a “strand”, forming a single- (ss oligo), or where two strands are annealed to each other, a double-stranded (ds oligo) nucleotide sequence.

Whenever an oligonucleotide is represented by a sequence of letters (upper or lower case), such as “ATGC” or “AGCU,” it will be understood that the nucleotides are in 5′→e3′ order from left to right and that “A” denotes deoxyadenosine, “T” denotes deoxythymidine, “G” denotes deoxyguanosine, “C” denotes deoxycytidine, and “U” denotes uracil. Besides conventional nucleotides (A, G, C, T, U), modified nucleotides e.g. K-2′-deoxyribose, P-2′-deoxyribose, 2′-deoxyinosine, 2′-deoxyxanthosine or nucleotides with nucleobase analogs may be used e.g., inosine, or 5-methylisocytosine, or 3-nitropyrrole, 5-nitroindole, pyrrolidine, 4-nitroimidazole, 4-nitropyrazole, 4-nitrobenzimidazole, 4-aminobenzimidazole, 5-nitroindazole, 3-nitroimidazole, 5-aminoindole, benzimidazole, 5-fluoroindole, indole, methylisocarbostyril, pyrrolopyrimidine 7-propynylisocarbostryril. The terminology and atom numbering conventions follow those disclosed in Strachan and Read, Human Molecular Genetics 2 (Wiley-Liss, New York, 1999). Usually, an oligonucleotide or polynucleotide comprises the four natural nucleosides (e.g., deoxyadenosine, deoxycytidine, deoxyguanosine, deoxythymidine for DNA or their ribose counterparts for RNA) linked by phosphodiester or by peptidyl linkages or by phosphorothioate linkages; however, they may also comprise non-natural nucleotide analogs e.g., including modified bases, sugars, or internucleosidic linkages.

Oligos designed for synthetizing a DNA polynucleotide are typically composed of bases (nucleosides) of a DNA. Specifically, an ss oligo used for a ds DNA assembly is DNA oligo, or comprises a DNA backbone. Oligos which are used as an auxiliary tool in the synthetization process and thus not incorporated in a target ds polynucleotide can be composed of artificial or chimeric or hybrid constructs e.g., comprising one or more DNA bases and/or one or more RNA bases and/or one or more artificial (non-natural) bases, such as one or more XNA bases.

The size of a ss oligo typically ranges between 6 and 26 nucleotides (nt), but may be longer e.g., between 6 and 220 nt, such as at least 6, 7, 8, 9, 10, 11, 12, 13, 14, 15, 16, 17, 18, 19, 20, 21, 22, 23, 24, 25, 26, or 27, up to 220, 210, 200, 190, 180, 170, 160, 150, 140, 130, 120, 110, 100, 90, 80, 70, 60, 50, 40, 30, 29, 28, 27, or 26 nt.

Ss oligos can be produced using chemical synthesis methods e.g., by synthesizing the oligonucleotide sequence from monomer-phosphoramidites, dimer-phosphoramidites (Neuner, Cortese, and Monaci 1998) or trimer-phosphoramidites (Sondek and Shortle 1992), mixture of monomer-phosphoramidites, mixture of dimer-phosphoramidites, mixture of trimer-phosphoramidites or their combination thereof.

In some embodiments, ss oligos are produced and purified from naturally-occurring sources, or synthesized in vivo, within the cell undergoing in vivo mutagenesis using any of a variety of well-known enzymatic methods e.g., as described in Farzadfard et al. (2014). Specifically, enzymes that synthesize soft-randomized ss oligos include, but are not limited to low fidelity DNA polymerase proteins or low fidelity reverse transcriptase proteins which incorporate mismatching nucleotides during synthesis with high frequency. Alternatively, mismatching nucleotides are incorporated into the ss oligos with a higher frequency by the DNA polymerases or reverse transcriptases due to the presence of chemical substances, which are well-known to those skilled in the art.

A ds oligo is understood as a linear polymer of nucleotide dimers. Dimers making up oligonucleotides comprise complementary nucleotides bound by way of a regular pattern of monomer-to-monomer interactions, such as Watson-Crick type of base pairing, base stacking, Hoogsteen or reverse Hoogsteen types of base pairing, wobble base pairing, or the like.

The size of a ds oligo such as a starter ds oligo described herein, typically ranges between 6 and 26 base pairs (bp), but may be longer. Ds oligonucleotides described herein may range in size between 6 and 200 bp, e.g., between 6 and 200 bp, such as at least 6, 7, 8, 9, 10, 11, 12, 13, 14, 15, 16, 17, 18, 19, 20, 21, 22, 23, 24, 25, 26, or 27, e.g., up to 220, 210, 200, 190, 180, 170, 160, 150, 140, 130, 120, 110, 100, 90, 80, 70, 60, 50, 40, 30, 29, 28, 27, or 26 bp.

A ds assembly such as a ds oligo or ds polynucleotide, may or may not comprise one or two overhangs, each overhang being understood as a single-stranded part of the ds oligo. Typically, the length of a ds assembly is understood as the length of the ds part of the ds assembly.

A ds assembly of a certain length is also referred to as a ds polynucleotide. A ds polynucleotide is typically understood as being composed of a number of ss and/or ds oligos.

The term “extender ss oligo (ExtON)” refers to a ss oligonucleotide which comprises a sticky terminal-sequence of at least 2 nucleotides on one side, and an identifiable tail on the other side. The ExtON may comprise or consist of an oligonucleotide sequence, e.g., comprising a DNA and/or RNA and/or artificial nucleotide sequence.

For example, an ExtON described herein may be composed of DNA, RNA, or a chimeric or hybrid nt sequence. In particular an ExtON described herein comprises any one or more of a DNA backbone, an RNA backbone or a chimeric or hybrid backbone. As commonly known, a nucleic acid is composed of an alternating chemical phosphate and sugar backbone. In between a sugar-phosphate backbone are the nucleotide bases. A DNA backbone is highly similar to an RNA backbone. The difference is that RNA backbone contains the sugar ribose, while DNA backbone contains the sugar deoxyribose. A chimeric or hybrid backbone may comprise the sugar ribose and/or the sugar deoxyribose and/or an artificial sugar such as a ribose or deoxyribose derivative.

The sticky sequence of an ExtON is typically a DNA sequence, which may hybridize to a DNA building block of a target DNA polynucleotide. However, the sticky sequence may comprise one or more heterologous nucleotides such as RNA and/or XNA nt.

A set of ExtONs is typically understood as a series of different ExtONs, e.g., a library of ExtONs, such as comprising a variety of ExtONs to cover a diversity of sticky sequences and an identifiable tail. According to a specific aspect, the identifiable tail comprises a unique molecular identifier (UMI) that is common to all ExtONs in the set or library.

Specifically, ExtONs are not part of a target ds polynucleotide but serve as a tool in DNA synthesis by providing additional bases to enable enzymatic ligation, such as the closure of a nick.

Specific ExtONs described herein are biotinylated.

As used herein, biotinylation refers to a method of covalently attaching one or more biotin molecules to a nucleic acid, such as an ExtON. ExtONs may be biotinylated by suitable methods well-known in the art; preferably it is a method of chemical biotinylation. ExtONs can be readily biotinylated in the course of synthesis by phosphoramidite methods well-known in the art, which use biotin phosphoramidite.

ExtONs described herein may be conjugated to a fluorophore by suitable chemical and enzymatic methods well-known in the art. Exemplary methods used for the fluorescent labeling of nucleic acids may employ a method for enzymatic labeling of DNA with fluorescent dyes e.g., using a Thermo Fisher's ARES DNA labeling kit, which employ a two-step method for enzymatic labeling of DNA with fluorescent dyes. Further exemplary methods may employ a chemical method for labeling nucleic acids without enzymatic incorporation of labeled nucleotides e.g., using a ULYSIS Nucleic Acid Labeling Kit. Further exemplary methods may employ chemical labeling of amine-terminated oligonucleotides to prepare singly labeled fluorescent oligonucleotide conjugates e.g., using an Alexa Fluor Oligonucleotide Amine Labeling Kit. Further exemplary methods may employ DNA arrays/microarrays and other hybridization techniques.

Specific ExtONs may be linked to one or more quenchers, e.g., substances that absorb excitation energy from a fluorophore, by suitable methods well-known in the art. Examples of quenchers include but are not limited to Dabsyl (dimethylaminoazobenzenesulfonic acid), Black Hole Quenchers, Qxl quenchers, Iowa black FQ, Iowa black RQ and IRDye QC-1.

The term “barcode” as used herein refers to a unique sequence region of an oligonucleotide that can be used to identify, track, or cross-reference the oligonucleotide sequence to a specific entity such as an oligo sequence, a nucleic acid molecule, cell, well in a microtiter or culture plate, or other application.

The barcode can be a nucleotide sequence of any length capable of being a unique identifier in the particular system. The barcode sequence can be any length, including 4-50 nucleotides, 4-20 nucleotides, 8-20 nucleotides, or any nucleotide lengths within the specified ranges. In one aspect the barcode region is a Unique Molecular Identifier (UMI). Specific UMIs are a type of molecular barcoding that provides error correction and increased accuracy during sequencing. UMI molecular barcodes are typically short, unique sequences used to tag molecules in a library. The same UMI can be used as an identifier of a group of variety of substances, such as different oligos, if all of the group members comprise the same UMI. Specifically, a barcode can be composed of e.g., a nucleotide sequence, such as comprising any one or more of DNA, RNA, synthetic nucleic acids such as comprising Xeno nucleic acids (XNAs), synthetic nucleic acid analogues, or chemically modified nucleotides, or a combination of anyone of the foregoing.

Where a barcode identifies an identifiable tail that is common to a set of ss oligos (e.g., ExtONs), it is particularly understood that the barcode of all of the ss oligos within the set is the same.

The terms “5′-end”, “3′-end”, “5′-terminal sequence”, “3′-terminal sequence” of a single stranded nucleotide sequence is herein understood to refer to the side or orientation of an end of the single stranded nucleotide sequence, and specifically understood in the following way.

The 5′-end of a single stranded nucleotide sequence is understood to comprise or consist of the 5′-terminal region e.g., a part of the ss oligo or polynucleotide, such as e.g., comprising or consisting of at least 1, 2, 3, 4, 5, 6, 7, 8, 9, 10, nucleotides or more e.g., at least 10%, 20%, 30%; 40%, 50%, 60%, 70%, 80%, 90%, 95%, 96%, 97%, 98%, 99%, up to or close to 100% of the full length ss molecule, which 5′-terminal region is including the 5′-terminus, i.e. the 5′-terminal nucleotide.

The 3′-end of a single stranded nucleotide sequence is understood to comprise or consist of the 3′-terminal region e.g., a part of the ss oligo or polynucleotide, such as e.g., comprising or consisting of at least 1, 2, 3, 4, 5, 6, 7, 8, 9, 10, nucleotides or more e.g., at least 10%, 20%, 30%; 40%, 50%, 60%, 70%, 80%, 90%, 95%, 96%, 97%, 98%, 99%, up to or close to 100% of the full length ss molecule, which 3′-terminal region is including the 3′-terminus, i.e. the 3′-terminal nucleotide.

The 5′-end of a double stranded nucleotide sequence is understood to comprise or consist of the 5′-terminal region of the leading strand e.g., a part of the leading strand of the ds assembly (e.g., a ds oligo or polynucleotide), such as e.g., comprising or consisting of at least 1, 2, 3, 4, 5, 6, 7, 8, 9, 10, nucleotides or more e.g., at least 10%, 20%, 30%; 40%, 50%, 60%, 70%, 80%, 90%, 95%, 96%, 97%, 98%, 99%, up to or close to 100% of the full length ds molecule, which 5-terminal region is including the 5′-terminus, i.e. the 5′-terminal nucleotide.

The 3′-end of a double stranded nucleotide sequence is understood to comprise or consist of the 3′-terminal region of the leading strand e.g., a part of the leading strand of the ds assembly (e.g., a ds oligo or polynucleotide), such as e.g., comprising or consisting of at least 1, 2, 3, 4, 5, 6, 7, 8, 9, 10, nucleotides or more e.g., at least 10%, 20%, 30%; 40%, 50%, 60%, 70%, 80%, 90%, 95%, 96%, 97%, 98%, 99%, up to or close to 100% of the full length ds molecule, which 3′-terminal region is including the 3′-terminus, i.e. the 3′-terminal nucleotide.

It is specifically understood that a nucleotide sequence comprises both, a 5′-end and a 3′-end.

The term “overhang” as used herein refers to a stretch of unpaired nucleotides at an end of a ds NA building block which is extending the double stranded part of the ds NA building block. These unpaired nucleotides can be in either of the leading or lagging strand of a doubled stranded NA building block, creating either 3′ or 5′ overhangs. Whereas in a blunt-ended molecule, both strands terminate in a base pair, a non-blunt end creates an overhang.

An overhang is specifically characterized by a ss terminal stretch of one or more nucleotides that can be reactive to hybridize with a matching ss sequence of a reaction partner (such as a ss NA building block e.g., an ExtON, or a ds NA building block), which ss terminal stretch is extending the double stranded part of the ds NA building block. In particular, the overhang is reactive insofar that it is capable of hybridizing with another ss oligo or overhang that comprises a matching sequence. A reactive overhang is herein also understood to comprise or consist of a reactive, thus “sticky” sequence or to provide a “sticky end” of the molecule.

An overhang may consist of one nucleotide, or can be longer. An overhang may comprise or consist of at least any one of 1, 2, 3, 4, 5, 6, 7, 8, 9, 10, 11 or 12 nucleotides. An overhang is typically not more than half of a ds NA building block length. For example, if said ds NA building block is a ds oligo that is 6 nucleotides long (in the ds part of the oligo), the overhang is typically not more than 3 nucleotides long, meaning the overhang can also be 1 or 2 nucleotides long. According to a specific example, if said ds oligo is 24 nucleotides long (in the ds part of the oligo), the overhang is typically not more than 12 nucleotides long, meaning it can also be 1, 2, 3, 4, 5, 6, 7, 8, 9, 10 or 11 nucleotides long.

According to a specific example of a ds assembly, a sticky sequence of an overhang is at least 2, 3, 4, 5, or 6 nt long, to match the sticky terminal sequence of an ExtON. Upon assembly of a ds NA building block with an overhang with another NA building block through hybridization of the overlap with a sticky sequence of the other NA building block, the overhang can be considered as being integrated within or part of an extended ds NA building block.

Where the overhang is at the 5′-end of the ds NA building block and is part of the leading strand, the overhang comprises the 5′-terminal region including the 5′-terminus of the leading strand.

Where the overhang is at the 3-end of the ds NA building block and is part of the leading strand, the overhang comprises the 3-terminal region including the 3-terminus of the leading strand.

Where the overhang is at the 5′-end of the ds NA building block and is part of the lagging strand, the overhang comprises the 3-terminal region including the 3-terminus of the lagging strand.

Where the overhang is at the 3-end of the ds NA building block and is part of the lagging strand, the overhang comprises the 5′-terminal region including the 5′-terminus of the lagging strand.

According to a specific example, a ds assembly can be composed of three ss oligos which are annealed and/or hybridized to form overhangs on both sides of the ds assembly e.g., with two overhangs of the leading strand, or with two overhangs of the lagging strand.

According to another specific example, a ds assembly can be composed of four ss oligos which are annealed and/or hybridized to form overhangs on both sides of the ds assembly e.g., with one overhang of the leading strand, and with one overhang of the lagging strand.

Where the overhang on the leading strand includes the 5′-terminus of the leading strand, the overhang on the lagging strand will include the 5′-terminus of the lagging strand, which results in one overhang on the 5′-end of the ds assembly, and one overhang on the 3-end of the ds assembly.

Where the overhang on the leading strand includes the 3-terminus of the leading strand, the overhang on the lagging strand will include the 3-terminus of the lagging strand, which again results in one overhang on the 5′-end of the ds assembly, and one overhang on the 3-end of the ds assembly.

Specifically, an overhang may comprise a sticky sequence or sticky end.

The term “sticky sequence” as used herein refers to a ss stretch of two or more nucleotides that is reactive to hybridize with a matching ss sequence of a reaction partner, such as another ss oligo or ss polynucleotide, or a ds NA building block with an overhang.

A sticky sequence within a ss oligo or an ExtON is typically positioned within a terminal sequence e.g., the 5′- or 3′-terminal sequence, in particular including the terminus. As described herein, the sticky sequence of an ExtON specifically matches the sequence of an overhang of the ds assembly, such as to provide an extended ds assembly e.g., an extended ds oligo which comprises the ds assembly and said ExtON that is hybridized to the matching overhang of the ds assembly.

A sticky sequence within an overhang allows assembly of a ds NA building block with another ds NA building block in the direction of the overhang. Where an intermediate comprises two overhangs i.e., on both of its ends, such intermediate is designed to assemble with further ds NA building blocks in both directions.

The term “sequence of interest” or “SOI” as used herein refers to the desired nucleotide or base pair sequence of the target ds polynucleotide which is to be produced in one or more assembly tiers. The SOI may serve as a DNA template according to which a target ds polynucleotide is synthetized.

The term “tail” with respect to a ss oligo refers to a 5′- or 3′-terminal part of the ss oligo including the respective terminus, but not both of the 5′- or 3′-terminal part. A tail may consist of a nucleotide sequence or be a derivative thereof, such as a fusion or conjugation product that comprises a nucleotide sequence and other molecular structures or artificial compounds. The length of an oligo tail varies and will depend on the full-length of the ss oligo: Specific examples refer to ss oligos comprising a tail with a length of at least 10%, 20%, 30%, 40%, 50%, 60%, 70%, 80%, or 90% of the nucleotide sequence of the ss oligo, and/or at least 2, 3, 4, 5, 6, 7, 8, 9, or 10 nucleotides.

The part of the ss oligo which is considered to be a “tail” is typically including only one of the 5′- or 3′-terminal nucleotide of the ss oligo, but not both. A tail that is positioned at the 5′-end of an ss oligo is typically including the 5′-terminal nucleotide. A tail that is positioned at the 3′-end of an ss oligo is typically including the 3′-terminal nucleotide.

Specifically, an ExtON comprises or is composed of a sticky terminal sequence that includes the terminus of one end, and the identifiable tail that includes the terminus of the other end. Specifically, the identifiable tail of an ExtON does not include the sticky terminal sequence of the ExtON. Specifically, there is no overlap of the identifiable tail and the sticky terminal sequence within an ExtON.

The term “identifiable tail” with respect to a ss oligo such as an ExtON refers to a tail of a ss oligo which comprises a molecular identifier that allows specific recognition and/or binding by a respective reader or ligand. To render an oligo tail identifiable, the molecular identifier can be incorporated into the ss oligo, or attached to the ss oligo. The molecular identifier may be a unique molecular identifier (UMI) specific for a sticky sequence of an ExtON or specific for a set of ExtONs.

The term “storage-stable” with respect to a NA building block of an ExtON of a certain nucleotide sequence refers to the maintenance of nucleotide quality during storage at room temperature for at least 12 months.

The term “target double stranded (ds) polynucleotide” refers to a polynucleotide having a predefined sequence, which is produced by the method provided herein. Specifically, said target ds polynucleotide is characterized by a sequence which is identical and/or corresponding to a SOI, or characterized by an intermediate sequence which is identical and/or corresponding to part of a SOI. If the target ds polynucleotide sequence has a sequence which is less than 100% identical to a SOI or the respective part of the SOI, the target ds polynucleotide is understood as a proxy ds polynucleotide that can be further modified to produce a ds polynucleotide that has a sequence which is identical and/or corresponding to the SOI or the respective part of the SOI.

The ligation of DNA starting with extremely short fragments has been a challenge in the prior art. The ability to start the tiered enzymatic assembly from short oligos, enables long-DNA synthesis in short timeframes.

Therefore, a novel method is provided for the enzymatic ligation of one or more short single stranded and/or double-stranded oligonucleotide DNA fragments, making use of ExtONs to enable assembly of complex DNA of arbitrary length starting from single stranded and/or double stranded oligonucleotides with a short length e.g., eight bases or smaller. The exemplary assembly process comprises enzymatic ligation. According to a specific example, 8-mer ss oligos are initially annealed to form double stranded fragments with sticky ends (overhangs) of four bases on both sides. The short fragments are then assembled in a hierarchical i.e., step-wise manner, by combining fragments with matching sticky ends.

The method described herein provides and advantage over prior art methods, because successfully ligating short oligos with the aid of ExtONs which are used as supplemental oligos to allow a ligase reaction to create a covalent bond connecting a nick between the 5′ and 3′ ends of the adjacent oligos within a DNA strand. Such ExtONs are specifically used as disposable tools that are removed from the assembly upon ligation.

According to a specific example, the following steps are carried out:
(A) Insertion of generic single stranded oligos (ExtONs) to provide additional base pairs and to thereby facilitating ligation of DNA fragments: For every ligation of a pair of short fragments, two ExtONs are introduced in the reaction with 5′ ends reverse complementary to the exposed overhangs, hence, the sticky ends are not ligated. The sole purpose of the generic ExtONs is to enable the ligation of the short fragments; hence, the ExtONs are not designed to be part of the final product. For this reason, they are being referred to as disposable.
(B) Optional: The 5′ end of the generic ExtONs is modified in such a way that its ligation to the neighboring strands is not possible, such as through a blocking moiety, to increase efficiency.
(C) Optional: Removal of the ExtONs. After the short fragments are ligated in pairs, the tiered assembly continues as described in prior art. After a series of tiered steps, a purification step is performed and the ExtONs are removed with the rest of the byproducts of the reaction.
(D) Optional: Targeted removal. The ExtONs can be designed to have a 3′ tail with specific requirements that allow their removal with standard biochemical procedures after they have served their original purpose (e.g., affinity capture, hybridization capture etc.)

According to a specific example, a template of the target sequence is partitioned into regular 8-mers and matching ExtONs are inserted in each annealing well that contains 4 oligos to be annealed (2 sense/anti-sense pairs per reaction), and are designed to hybridize with the flanking oligos' 5′ overhangs, providing the extra bp that facilitate an enzymatic DNA ligation reaction to close the central nick composed by each reactions' two pairs of 8-mers.

For example, the ExtONs are designed in a specific manner to allow their efficient removal after the first tiered ligation reaction step. Irrespective of the removal method, all of them are designed and manufactured to harbour a generic, standard section that is identical to each ExtON, and a specific overhang i.e., a sticky sequence. The total number of ExtONs with different 4-mer sticky sequences is 256. Such ExtONs are amenable to store within an oligo biobank for continuous usage throughout automated assembly routine.

According to a specific example, after Tier 1 ligation, the reaction well temperature is raised in a controlled, automated manner, to separate the ExtONs from the rest of the fragments, enabling specific ExtON capture and removal steps. There are several possible solutions for the removal step.
a. Standard purification. Introducing a blocking or other “inert” modification to a generic oligo of regular size (e.g., a regular 8-mer) to prevent ligation but allow hybridization of the overhang section with its complementary counterpart. In this way, after the first round of purification the ExtONs will be removed. This does not require additional removal mechanism other than the current standard purification (SPRI-bead size-specific, sequence-independent purification).
b. Temperature driven selection. Using regular oligos and increasing the temperature through automated controlled temperature modulation of the system just enough to denature the shorter section, so that the ExtONs will detach from the ligated dimer (e.g., 16 mer) right upon Tier 1 ligation conclusion. Then, using a generic method of dsDNA purification is sufficient, since the remaining single-stranded oligos will be removed.
c. Barcode modification. The designed ExtONs have a standardized barcode, composed of DNA, XNA synthetic nucleic acid analogues, or chemically modified nucleotides, on one end that can be captured, cleaved, ligated or generally “controlled” by a specific mechanism. After the first ligation is performed, the temperature is raised to a threshold value to dissociate and denature the ExtONs, which are captured by a “bait” reagent, moiety or device according to the associated “prey” design, for instance by a ssDNA fragment coated surface that inversely matches the barcode sequence. This approach is simple to automate even in a microfluidic setting and the capture is efficient. A large space of target capture approaches can be employed and includes possibilities to include click chemistry, covalent chemistry, XNA-based ligation/cleavage strategies alongside regular sense/anti-sense hybridisation approaches toward the identification of the most efficient methodology.
d. Affinity probe. This approach represents a similar mechanism to the one described in section (c), but instead of the sequence barcode tag, an affinity moiety, for example biotin, is attached to the ExtON, which can be therefore separated through an affinity-based target capture method e.g., combined with support attachment, be it magnetic or through other reversible bonding, on beads or solid surface structure, which can include capillary walls or 3-dimensional chambers in a microfluidic setup. Depending on the affinity-capture method chosen, this approach can be automated, even in a microfluidic setting, and the capture is efficient.

According to a specific example described herein, an ExtON can be removed due to the inherent limitations a DNA ligase that does not perform ligation with a limited number of base-pairs adjacent to the nick site in the specific setting. Even if the ExtON is phosphorylated, it will itself not form a covalent bond with other oligos within the ds assembly, and it can always be denatured with increased temperature, and captured through above-mentioned strategies for “disposal”, not to jeopardize downstream assembly performance and product purity.

The foregoing description will be more fully understood with reference to the following examples. Such examples are, however, merely representative of methods of practicing one or more embodiments of the present invention and should not be read as limiting the scope of invention.

### EXAMPLES

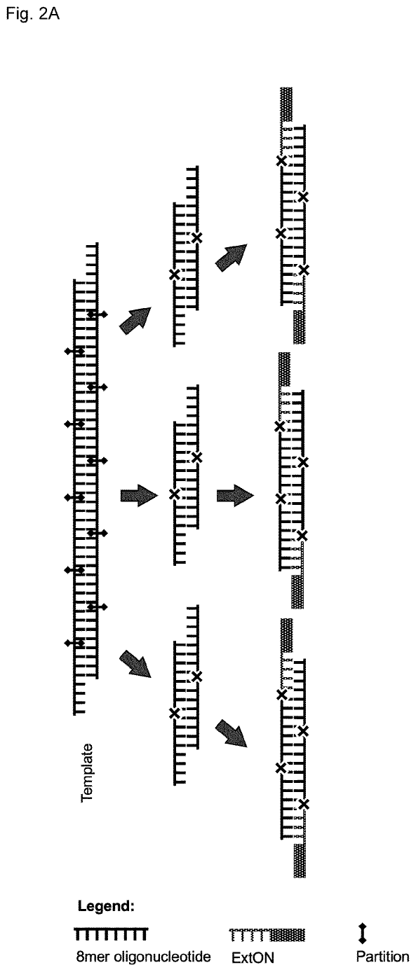

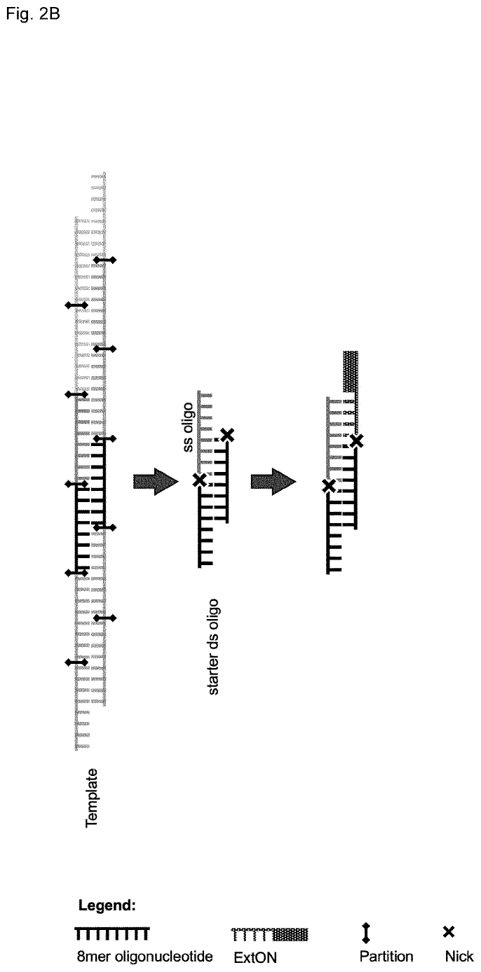

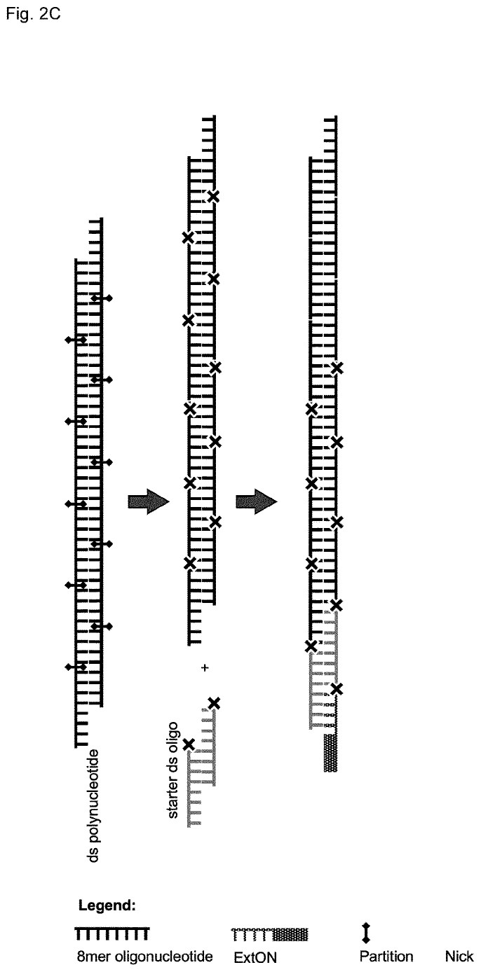

The use of disposable Extention OligoNucleotides, “ExtONs” (FIG. 2) is described in the following Example, as ligation enabling support oligos, which are to be inserted in the partitioning of the sequence, to be included in the initial annealing and first tier of ligation reactions, and to be removed afterwards, restoring the standard tiered assembly.

In the following example, the method proceeds as follows: the standard sequence is partitioned into regular 8-mers (see FIG. 2). A pair of matching ExtONs (blocks) is inserted in each annealing well that contains 4 oligos to be annealed (2 sense/anti-sense pairs per reaction). The ExtONs are designed to hybridize with the 5′ overhangs of the flanking oligos', providing the extra base pairs (bp) that are needed to enable the enzymatic DNA ligation reaction to close the central nick composed by each reaction's two pairs of 8-mers.

### Example 1. Comparison of Assemblies of Short Fragments with and without the Use of ExtONs

In this example, the role of ExtONs in the enzymatic assembly of short oligonucleotides is shown by comparing the results of two assemblies performed under completely analogous conditions. In the first assembly (assembly 1), ExtONs are added to the assembly reaction, whereas in the second (assembly 2) they are not added. The results show that in the absence of ExtONs the assembly fails in most cases, producing only a few bands in the polyacrylamide gel electrophoresis (PAGE).

### Method

A. A total of 32 single stranded 8-mer oligonucleotides (SEQ ID NOs:1-32) are designed to form 16 double stranded oligos with 4 base overhangs. The 5′ end of each oligo contains a phosphate modification. Another set of 16 ExtONs (SEQ ID NOs:33-48) is designed to have 5′ end sequences matching the overhangs of the oligo dimers to enable the ligation, but no phosphate modification is included on ExtONs. The total length of each ExtON is 12 bases. All oligos are adjusted to an equal concentration of 150 μM.
B. Annealing is performed in a 96 Well PCR Plate to obtain double stranded DNA fragments. Oligo pairs are mixed in T4 DNA ligase reaction buffer (New England BioLabs Inc., GB) and T4 Polynucleotide Kinase, or PNK (New England BioLabs Inc., GB).
C. The choice of T4 buffer over T3 buffer is due to the impossibility of heat-inactivate PEG8000, present in the latter. Heat inactivation is part of the analytics in the following composition in each well:
   a. 4.5 μL of each oligo
   b. 1 μL of T4 buffer
   c. 1 μL PNK
   A temperature ramp is performed by heating the samples to 60° C. for 5 minutes and decreasing by 1 degree every minute to 4° C.
D. The annealed oligo dimers are then ligated in pairs by using T3 ligase (New England BioLabs Inc., GB) (3000 U/μL) with the following composition in each well:
   a. 0.5 μL of T3 ligase
   b. 1.2 μL of ATP (10 mM)
   c. 0.6 μL of T4 buffer
   d. Differential step for the two assays:
      i. 1.35 μL of each of the two ExtONs for assembly 1
      ii. Nothing for assembly 2
   e. 3 μL of each of the two annealed oligo dimers
   f. Differential step for the two assays:
      i. 1 μL of water for assembly 1
      ii. 3.7 μL of water for assembly 2
E. The ligations are performed by incubating the mixtures for 60 minutes at 4° C.
F. The resulting product is analyzed by using a high percentage, 8 molar urea PAGE.

### Results

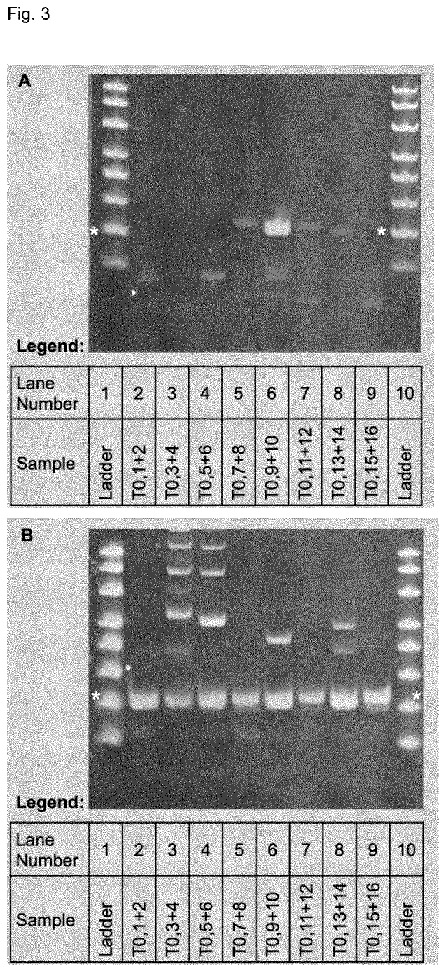

As it appears clearly from FIG. 3, that the target length of 16 bp is consistently achieved only in the case in which the extension oligonucleotides (ExtONs) are added to the reaction mix (FIG. 3B). This proves that the presence of the ExtONs is an enabler of the reaction. Without them, the ligase enzyme did not sufficiently perform the reaction in this setting (FIG. 3A). The procedure also shows clearly that the ligation is performed only on the central fragments in the array, which means that it is possible to continue the ligation beyond the first tier by combining the consecutively ligated products as it is done in the standard prior art procedure.

### Example 2. Temperature-Controlled Assembly Followed by Bead Purification

This example is the continuation of the setup described in Example 1, however, this time the assembly is carried on to higher tiers. The first ligation is performed at a lower temperature to improve the stability of the hydrogen bonds between the two DNA strands. After the first incubation period, the temperature is again raised at a higher value, so that the ExtONs can detach from the target oligos, which can therefore find the proper partner in the successive ligations.

There are in total 4 steps of ligation this time, the first one is analogous to the one described in Example 1, with a difference in temperature. Then the temperature is further raised for the successive tiers to allow additional reactions.

### Method

A. Same steps of Example 1, at 16° C. with incubation of 90 minutes.
B. Deactivation of T3 ligase by incubating for 20 minutes at 70° C.
C. 5 μL of each pair of ligated products, with the addition of T7 ligase (New England BioLabs Inc., GB) (0.75 μL) and ATP (2 μL).
D. Increased incubation temperature to 24° C. and incubated for 90 minutes.
E. Repeated incubation after pooling again each product of the preceding reaction two more times.
F. Perform bead enrichment MagMAX™ DNA Multi-Sample Ultra 2.0 Binding Beads (LTC Product Nr. A36579C) on 30 μL of final product after 4 ligation steps. 1.66 μL binding buffer to select the target size (~130 bp final molecule length) and 15 μL of elution volume.
G. Final product, before and after bead enrichment, is analyzed with capillary electrophoresis (Fragment Analyzer System).

### Results

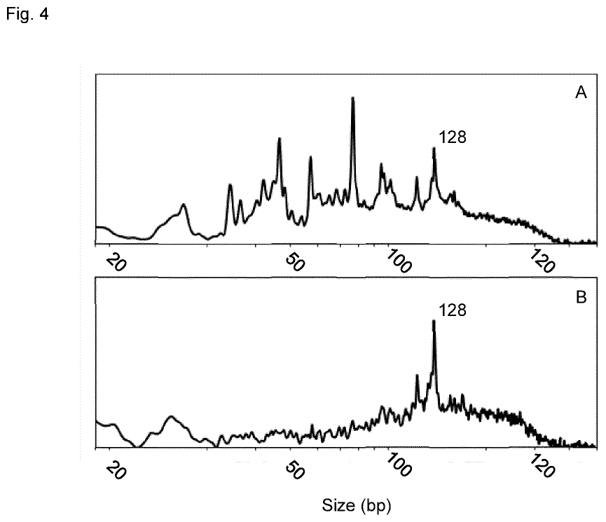

As clearly visible from the electropherogram in FIG. 4, the target length is obtained after 4 steps of ligations. The increase of temperature after the first tier of ligation ensured that the chance of an interference of the ExtONs was minimized. After bead enrichment, each shorter molecule, including the ExtONs, is removed from the reaction. The final products can then be used to assemble molecules of arbitrary length as described in prior art.

### Example 3. Affinity-Probe Capture of ExtONs with the Use of Poly-A Tails

In the previous examples, it was shown how the ExtONs can enable the enzymatic assembly of short oligos, and the modulation of the reaction temperature makes sure that the reaction can continue to higher tiers. To ensure the overall efficiency of the ligation, the ExtONs are removed after the first tier of ligation. Since it is difficult to perform length/based enrichment for fragments that are shorter than 100 bp, a method to remove the ExtONs was devised using an affinity probe. There are several methods that can be implemented. All of them require the identifiable tail of the ExtONs to be designed in a way that allows capture. In this specific case, the ExtONs are designed to have a ligation-specific initial 4 bases on the 5′ end. For graphical reason these specific bases are identified with n, wherein n is any one of the standard nucleotide bases, A, G, C, T, or U, and the additional 16 bases (for a total of 20 bases) are composed of A (DNA backbone: SEQ ID NO:49; RNA backbone: SEQ ID NO:50).

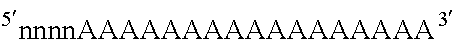

Wherein
n at positions 1, 2, 3, 4 is one nucleotide independently selected from the standard nucleotide bases A, G, C, T, or U.

In this way, the 3′-poly-A tail can bind to commercially available magnetic beads designed with poly-T affinity probe. These beads allow the extraction of the unreacted, unused product from the reaction, and then continue the process with the leftover.

### Method

A. Perform the first ligation as described in the previous Examples.
B. Use Oligo d(T)₂₅ Magnetic Beads (New England BioLabs Inc., GB) purification protocol to extract the unreacted support product.
C. Continue the ligations as described in Example 2.

### Results

There is a great increase of the performance of the ligation by this process, since there is a more balanced stoichiometry to the ongoing reaction, preventing mis-ligations at higher tiers.

### Example 4. Using a Single ExtON to Ligate a Single-Stranded Oligonucleotides to a Double-Stranded Oligonucleotide

There can be limitations of the enzymes in the ligation of short fragments to create a covalent bond in the position of the ligation site when the number of base pairs around the nick is less than 5. By this Example it is shown that it is possible to ligate a double stranded oligo to a single stranded oligo by using a single ExtON.

Therefore, a ligation by having 3 single stranded oligos and one ExtON can be performed and proceeding as in Examples 1 and 2, but with the following modifications in the initial procedure:
A. Anneal a single pair of oligos with 8 bases designed in a way of leaving 5′ overhangs with 4 bases.
B. Combine the annealed pair with the additional single-stranded oligo in such a way to hybridize the sticky ends together. A double stranded oligo with overhangs on both ends and a nick in the overlapping portion is obtained.
C. Combine the resulting product with the ExtON, which will have a sticky end to hybridize to the 5′ end of the double stranded oligo.
D. Proceed with the successive ligation steps.

The final product is a hybridized structure of a 16 base oligonucleotide and an 8-base oligonucleotide (SEQ ID NOs:51 and 52 of leading and lagging strand, respectively).

### Example 5. ExtONs Containing a 5′ Modification that Prevents Phosphorylation

This example is an alternative to the ones described in Examples 1 and 2, with the only difference on the nature of the ExtONs: This time they contain a 5′ phosphorylation-preventing modification.

Although the nature of the enzyme might not allow efficient ligation of the nick when the number of base pairs around the ligation site is less than 5, some ligation events can still happen because of contingent circumstances. This means that some ExtONs can be ligated to the ds fragment at the first tier of ligation or beyond, decreasing the overall efficiency of the process. For this reason, we provide a modification on the 5′ end, the sticky end of the ExtON. This modification prevents phosphorylation, making sure that no byproduct is produced because of the ExtONs being ligated.

### Method

A. A total of 32 single stranded 8-mer oligonucleotides (SEQ ID NOs:1-32) are designed to form 16 double stranded oligos with 4 base overhangs. The 5′ end of each oligo contains a phosphate modification. Another set of 16 ExtONs is designed to have 5′ end sequences matching the overhangs of the oligo dimers to enable the ligation (SEQ ID NO:54).

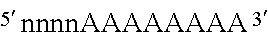

Wherein
   n at position 1 is any one of the standard nucleotide bases, A, G, C, T, or U, which has been chemically modified to prevent unwanted 5′ phosphorylation and terminal ligations by replacing the respective terminal A, G, C, T or U nucleoside's reactive 5′-OH with a dimethyloxytrityl (DMT) group, generating a 5′-DMTO cap, or using 5′ Inverted Dideoxy-T, or 5′-OMe-dT for terminal T DNA backbone;
   n at positions 2, 3, 4 is one nucleotide independently selected from the standard nucleotide bases A, G, C, T, or U.
The total length of each ExtON is 12 bases. All oligos are adjusted to an equal concentration of 150 μM.
B. Annealing is performed in a 96 Well PCR Plate to obtain double stranded DNA fragments. Oligo pairs are mixed in T4 DNA ligase reaction buffer (New England BioLabs Inc., GB) and T4 Polynucleotide Kinase, or PNK (New England BioLabs Inc., GB).
C. The choice of T4 buffer over T3 buffer is due to the impossibility of heat-inactivate PEG8000, present in the latter. Heat inactivation is part of the analytics in the following composition in each well:
   a. 4.5 μL of each oligo
   b. 1 μL of T4 buffer
   c. 1 μL PNK
D. A temperature ramp is performed by heating the samples to 60° C. for 5 minutes and decreasing by 1 degree every minute to 4° C.
E. The annealed oligo dimers are then ligated in pairs by using T3 ligase (New England BioLabs Inc., GB) (3000 U/μL) with the following composition in each well:
   a. 0.5 μL of T3 ligase
   b. 1.2 μL of ATP (10 mM)
   c. 0.6 μL of T4 buffer
   d. 1.35 μL of each of the two ExtONs
   e. 3 μL of each of the two annealed oligo dimers
   f. 1 μL of water
F. The ligations are performed by incubating the mixtures for 60 minutes at 4° C.
G. The resulting product is analyzed by using a high percentage, 8 molar urea PAGE.

### Results

The overall yield of the reaction after the bead enrichment step is increased as compared to the reaction with the non-modified ExtONs.

### Example 6. ExtONs Containing Uracil Base

This example is an alternative to the ones described in Examples 1 and 2, with the only difference being in the nature of the ExtONs: This time they contain a single Uracil base in their sequence, otherwise they are of the same composition as those described in the previous examples.

According to a particular embodiment, an ExtON may comprise a DNA backbone, a sticky sequence, and the first nucleotide following the sticky sequence at the 3′-end is an Uracil (“U”). Accordingly, the U can be the 5′ terminal nucleotide of the identifiable tail. For the purposes of this example, we provide the sequence referred to herein.

### Method

A. A total of 32 single stranded 8-mer oligonucleotides (SEQ ID NOs:1-32) are designed to form 16 double stranded oligos with 4 base overhangs. The 5′ end of each oligo contains a phosphate modification. Another set of 16 ExtONs is designed to have 5′ end sequences matching the overhangs of the oligo dimers to enable the ligation, but the ExtONs have an DNA backbone with a single Uracil (SEQ ID NO:53).

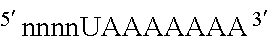

Wherein
   n at positions 1, 2, 3, 4 is one nucleotide independently selected from the standard nucleotide bases A, G, C, T, or U.
SEQ ID NO:53 according to WIPO ST26 standard: DNA

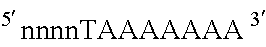

Wherein
   n at positions 1, 2, 3, 4 is one nucleotide independently selected from the standard nucleotide bases A, G, C, T, or U;
   T″ at position 7 is “uracil”.
The total length of each ExtON is 12 bases. All oligos are adjusted to an equal concentration of 150 μM.
B. Annealing is performed in a 96 Well PCR Plate to obtain double stranded DNA fragments. Oligo pairs are mixed in T4 DNA ligase reaction buffer (New England BioLabs Inc., GB) and T4 Polynucleotide Kinase, or PNK (New England BioLabs Inc., GB).
C. The choice of T4 buffer over T3 buffer is due to the impossibility of heat-inactivate PEG8000, present in the latter. Heat inactivation is part of the analytics in the following composition in each well:
   a. 4.5 μL of each oligo
   b. 1 μL of T4 buffer
   c. 1 μL PNK
D. A temperature ramp is performed by heating the samples to 60° C. for 5 minutes and decreasing by 1 degree every minute to 4° C.
E. The annealed oligo dimers are then ligated in pairs by using T3 ligase (New England BioLabs Inc., GB) (3000 U/μL) with the following composition in each well:
   a. 0.5 μL of T3 ligase
   b. 1.2 μL of ATP (10 mM)
   c. 0.6 μL of T4 buffer
   d. 1.35 μL of each of the two ExtONs
   e. 3 μL of each of the two annealed oligo dimers
   f. 1 μL of water
F. The ligations are performed by incubating the mixtures for 60 minutes at 4° C.
G. The resulting product is analyzed by using a high percentage, 8 molar urea PAGE.

### Example 7. RNA ExtONs

This example is an alternative to the ones described in Examples 1 and 2, with the only difference being in the nature of the ExtONs: This time they are entirely made out of RNA residues, with sequences in the sticky end that match the corresponding DNA counterparts.

### Method

A. A total of 32 single stranded 8-mer oligonucleotides (SEQ ID NOs:1-32) are designed to form 16 double stranded oligos with 4 base overhangs. The 5′ end of each oligo contains a phosphate modification. Another set of 16 ExtONs is designed to have 5′ end sequences matching the overhangs of the oligo dimers to enable the ligation, but the ExtONs have an RNA backbone (SEQ ID NO:50).

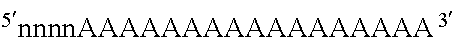

Wherein
   n at positions 1, 2, 3, 4 is one nucleotide independently selected from the standard nucleotide bases A, G, C, T, or U.
The total length of each ExtON is 12 bases. All oligos are adjusted to an equal concentration of 150 μM.
B. Annealing is performed in a 96 Well PCR Plate to obtain double stranded DNA fragments. Oligo pairs are mixed in T4 DNA ligase reaction buffer (New England BioLabs Inc., GB) and T4 Polynucleotide Kinase, or PNK (New England BioLabs Inc., GB).
C. The choice of T4 buffer over T3 buffer is due to the impossibility of heat-inactivate PEG8000, present in the latter. Heat inactivation is part of the analytics in the following composition in each well:
   a. 4.5 μL of each oligo
   b. 1 μL of T4 buffer
   c. 1 μL PNK
D. A temperature ramp is performed by heating the samples to 60° C. for 5 minutes and decreasing by 1 degree every minute to 4° C.
E. The annealed oligo dimers are then ligated in pairs by using T3 ligase (New England BioLabs Inc., GB) (3000 U/μL) with the following composition in each well:
   a. 0.5 μL of T3 ligase
   b. 1.2 μL of ATP (10 mM)
   c. 0.6 μL of T4 buffer
   d. 1.35 μL of each of the two ExtONs
   e. 3 μL of each of the two annealed oligo dimers
   f. 1 μL of water
F. The ligations are performed by incubating the mixtures for 60 minutes at 4° C.
G. The resulting product is analyzed by using a high percentage, 8 molar urea PAGE.

### Example 8. ExtONs are Digested with RNase H

This example describes the procedure of removal of RNA ExtONs, as described in Example 7, with the use of an RNase enzyme, here specifically RNase H (New England Biolabs GmbH). In an example as the one described in Examples 1 and 2, but where ExtONs are composed entirely from RNA base residues, ExtONs are digested, cleaved or degraded following conclusion of the ligation reaction using an enzymatic digest with RNase H enzyme, which recognizes and cleaves specifically only the phosphodiester bonds of the RNA ExtON, while leaving the DNA intact.

### Method

A. Same steps of Example 7
B. In a final step following conclusion of the ligation reaction (Example 1, Step E.), RNA ExtONs are digested, cleaved or degraded via RNase H treatment with the following composition
   a. 0.2 μg assembly reaction including RNA ExtONs
   b. 1 μl RNase H Reaction Buffer (10×)
   c. 0.1 μl (0.5 units) RNase H
   d. Up to 10 μl Nuclease-free H₂O
C. Incubate at 37° C. for 20 minutes

### REFERENCES

Horspool, D. R., Coope, R. J. N., Holt, R. A. (2010) Efficient assembly of very short oligonucleotides using T4 DNA Ligase. BMC Research Notes 3(291):1-9.
Farzadfard F, Lu T K. (2014) Synthetic biology. Genomically encoded analog memory with precise in vivo DNA writing in living cell populations. Science 346(6211):1256272.
Neuner, P., Cortese, R., & Monaci, P. (1998) Codon-based mutagenesis using dimer-phosphoramidites. Nucleic acids research 26(5), 1223-1227.
Sondek, J., Shortle, D. (1992) A general strategy for random insertion and substitution mutagenesis:substoichiometric coupling of trinucleotide phosphoramidites. Proceedings of the National Academy of Sciences 89(8), 3581-3585.

1. A method to produce a target double-stranded (ds) DNA polynucleotide by annealing single-stranded oligonucleotides (ss oligos) and ligating a ds assembly, comprising the following steps:
a) annealing at least 2 ss oligos comprising matching sequences for hybridization to produce a starter ds oligo with two overhangs, and hybridizing one of said overhangs with a nucleotide (NA) building block, thereby obtaining a ds assembly with two overhangs of at least 2 nucleotides (nt), which ds assembly comprises at least one nick in the ds part of the ds assembly;
b) hybridizing to the ds assembly at least one extender ss oligo (ExtON), which comprises a sticky terminal sequence of at least 2 nt on one side, and an identifiable tail on the other side, wherein the sticky terminal sequence is matching for hybridization with an overhang of the ds assembly, thereby obtaining an extended ds assembly;
c) ligating the nucleotides within the extended ds assembly by a ligation reaction, thereby resolving said at least one nick; and
d) following the ligating step c), removing said at least one ExtON from the extended ds assembly, thereby producing the target ds polynucleotide.

2. The method of claim 1, wherein in said step a):
i) at least four ss oligos are annealed and hybridized to produce a ds assembly comprising at least two nicks; or
ii) three ss oligos are annealed and hybridized to produce a ds assembly comprising one nick; or
iii) two ss oligos are annealed and hybridized to one ds oligo or ds polynucleotide to produce a ds assembly comprising two nicks.

3. The method of claim 1, wherein step a) comprises annealing and hybridizing four ss oligos, by annealing a first and a second pair of ss oligos, wherein each ss oligo pair comprises two ss oligos that comprise a part matching for annealing the ss oligos of the pair, and another part to obtain an overhang of at least 2 nt on each side upon annealing, and wherein one overhang of the first ss oligo pair is matching for hybridization with one overhang of the second ss oligo pair to produce said ds assembly.

4. The method of claim 1, wherein steps a) and b), and optionally step c), are carried out in one reaction containment.

5. The method of claim 1, wherein two ExtONs are used for hybridization to the ds assembly, wherein the sticky sequences of said two ExtONs are matching for hybridization with the two overhangs of the ds assembly, thereby obtaining an extended ds assembly that comprises an ExtON on each side.

6. The method of claim 1, wherein each of the ss oligos comprises at least 6 nt.

7. The method of claim 1, wherein the sticky sequence of said at least one ExtON is comprised of the 5′-terminal sequence of the ExtON, and the tail is comprised of or attached to the 3′-terminal sequence of the ExtON.

8. The method of claim 1, wherein the sticky sequence of said at least one ExtON is not phosphorylated and/or comprises a 5′ blocking modification that prevents ligation.

9. The method of claim 1, wherein the tail of said at least one ExtON comprises a unique molecular identifier (UMI).

10. The method of claim 1, wherein said at least one ExtON is removed by dehybridization and optionally isolating said at least one ExtON or the target ds polynucleotide.

11. The method of claim 1, wherein the ligation reaction is an enzymatic reaction and wherein the enzyme is a ligase, polymerase, or ribozyme.

12. The method of claim 1, which further comprises producing an assembly of the target ds polynucleotide and one or more DNA building blocks in one or more further steps, to synthetize a longer ds polynucleotide according to a DNA template.

13. A set of extender single-stranded oligonucleotides (ss oligo) (ExtONs), wherein each ExtON comprises a sticky terminal sequence of at least 2 nucleotides (nt) on one side, that is reactive to hybridize with a matching ss sequence of a double-stranded (ds) nucleotide (NA) building block, and an identifiable tail on the other side, wherein the identifiable tail comprises a unique molecular identifier (UMI) that is specific to the set of ExtONs and common to all ExtONs in the set, which set comprises a variety of ExtONs to cover a diversity of said sticky sequences.

14. The ExtON set of claim 13, wherein each ExtON is characterized by one or more of the following:
a) the sticky sequence of the ExtON consists of at least 2 nt;
b) the sticky sequence of the ExtON is comprised of the 5′-terminal sequence of the ExtON, and the tail is comprised of or attached to the 3′-terminal sequence of the ExtON;
c) the sticky sequence of the ExtON is not phosphorylated and/or comprises a 5′ blocking modification that prevents ligation;
d) the ExtON consists of an oligonucleotide of at least 8 nt; and
e) the ExtON consists of an oligonucleotide with a length of up to 30 nt.

15. The method of claim 1, wherein the NA building block is a ss oligo, a ds oligo or a ds polynucleotide.

16. The method of claim 4, wherein steps a) and b), and optionally c), are carried out in a pool reaction.

17. The method of claim 11, wherein the enzyme is any one of a T3, T4 or T7 DNA ligase, Taq DNA ligase, or a DNA polymerase.

18. The method of claim 13, wherein the variety of ExtONs covers a diversity of at least 10 different sticky sequences.

19. The method of claim 14, wherein the sticky sequence of the ExtON comprises a 5′ blocking modification that prevents phosphorylation.

20. The method of claim 14, wherein the ExtON consists of an oligonucleotide with a length of up to 20 nt.
